SEQUENCE LISTING (16 sequences)

SEQ ID NO: 1 - protein, 9 aa, Artificial Sequence
  CDR Sequence
YTFTDLYMK

SEQ ID NO: 2 - protein, 17 aa, Artificial Sequence
  CDR Sequence
DINPNNGDANYNQKFKG

SEQ ID NO: 3 - protein, 7 aa, Artificial Sequence
  CDR Sequence
ARRGGTY

SEQ ID NO: 4 - protein, 16 aa, Artificial Sequence
  CDR Sequence
RSSQSIVHSNGDTYLE

SEQ ID NO: 5 - protein, 7 aa, Artificial Sequence
  CDR Sequence
RVSNRFS

SEQ ID NO: 6 - protein, 9 aa, Artificial Sequence
  CDR Sequence
FQGSHLPWT

SEQ ID NO: 7 - protein, 8 aa, Artificial Sequence
  CDR Sequence
GYTFTDLY

SEQ ID NO: 8 - protein, 8 aa, Artificial Sequence
  CDR Sequence
INPNNGDA

SEQ ID NO: 9 - protein, 7 aa, Artificial Sequence
  CDR Sequence
ARRGGTY

SEQ ID NO: 10 - protein, 12 aa, Artificial Sequence
  CDR Sequence
SQSIVHSNGDTY

SEQ ID NO: 11 - protein, 3 aa, Artificial Sequence
  CDR Sequence
RVS

SEQ ID NO: 12 - protein, 9 aa, Artificial Sequence
  CDR Sequence
FQGSHLPWT

SEQ ID NO: 13 - protein, 444 aa, Artificial Sequence
  HC Sequence
EVKLQESGPELVKPGASVKMSCKASGYTFTDLYMKWVRQSHGRNLEWIGDINPNNGDANYNQKFKGKATLTVDKSSSTAYMQLNSLTSEDSAVYYCARRGGTYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNRYTQKSLSLSPGK

SEQ ID NO: 14 - protein, 219 aa, Artificial Sequence
  LC Sequence
DVLMTQTPLSLPVSLGDQASISCRSSQSIVHSNGDTYLEWYLQKPGQSLKLLIYRVSNRFSGVPDRFSGSGSGTDFTLKISRVEAEDLGLYYCFQGSHLPWTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 15 - protein, 114 aa, Artificial Sequence
  VH Sequence
EVKLQESGPELVKPGASVKMSCKASGYTFTDLYMKWVRQSHGRNLEWIGDINPNNGDANYNQKFKGKATLTVDKSSSTAYMQLNSLTSEDSAVYYCARRGGTYWGQGTTVTVSS

SEQ ID NO: 16 - protein, 112 aa, Artificial Sequence
  VL Sequence
DVLMTQTPLSLPVSLGDQASISCRSSQSIVHSNGDTYLEWYLQKPGQSLKLLIYRVSNRFSGVPDRFSGSGSGTDFTLKISRVEAEDLGLYYCFQGSHLPWTFGGGTKLEIK

(19) United States
(12) Patent Application Publication
(10) Pub. No.: US 2018/0312576 A1
(43) Pub. Date: Nov. 1, 2018

(54) ANTIBODIES TARGETING A MANNAN-BASED O-ANTIGEN OF K. PNEUMONIAE

(71) Applicant: ARSANIS BIOSCIENCES GMBH, Vienna (AT)

(72) Inventors: Valeria SZIJARTO, Wien (AT); Gábor NAGY, Sopron (HU); Katharina HARTL, Vienna (AT); Luis GUACHALLA, Wien (AT); Eszter NAGY, Vienna (AT)

(73) Assignee: ARSANIS BIOSCIENCES GMBH, Vienna (AT)

(21) Appl. No.: 15/768,656

(22) PCT Filed: Jun. 30, 2016
(86) PCT No.: PCT/EP2016/065301

§ 371 Date: Apr. 16, 2018

(30) Foreign Application Priority Data
Oct. 28, 2015 (EP) 15191877.8

(51) Int. Cl. C07K 16/12; G01N 33/569

(52) U.S. Cl. CPC C07K 16/1228; G01N 33/56916; C07K 2317/33; G01N 2333/26; C07K 2317/21; C07K 2317/24; C07K 2317/92

(57) ABSTRACT

The invention provides for an isolated antibody that specifically recognizes an epitope of the lipopolysaccharide (LPS) O3b-antigen structure of Klebsiella pneumoniae, which is a O3b-epitope incorporated in O3b-antigen comprising the structure of Formula (I), including one or more O3b-antigen mannose homopolymer repeating units, wherein Formula (I) is:

MeP→3)-α-D-Manp-(1→2)-α-D-Manp-(1→3)-α-D-Manp-(1→[3)-α-D-Manp-(1→2)-α-D-Manp-(1→3)-α-D-Manp-(1→]ₙ
wherein
MeP is methyl phosphate; and
n is 0-50.

The invention further provides for a pharmaceutical or diagnostic preparation comprising said antibody, and a method of producing said antibody.

### FIELD OF THE INVENTION

The invention refers to an isolated antibody that specifically recognizes an epitope of the lipopolysaccharide (LPS) O3b-antigen structure of Klebsiella pneumoniae, which is an O3b-epitope incorporated in O3b-antigen comprising a heretofore unidentified structure.

### BACKGROUND OF THE INVENTION

Klebsiella pneumoniae is an important enterobacterial pathogen responsible for nosocomial infections that cause significant morbidity and mortality. Multi-drug resistant (MDR) strains have recently emerged and spread globally, against which therapeutic options are limited. The current aim is to develop therapeutic monoclonal antibodies for the prevention and treatment of infections caused by MDR Klebsiella strains. The molecular target of the intended mAbs is the LPS O-antigen that is considered to be one of the few (if not the sole) antigen on the surface of Klebsiella.

Based on published epidemiology data (1,2) on O-type distribution, the majority of clinically relevant isolates belong to 4 serotypes, i.e. O1, O2, O3 and O5. O1 and O2 antigens are built up by homopolymers of galactoses (i.e. galactans), while O3 and O5 serogroups are composed of mannose homopolymers (i.e. mannans) (3).

The O3 serotype is characterised by the “classical” penta-mannose structure, shown in FIG. 1 (published in (3)). The penta-mannose structure of Klebsiella O3 antigen was elucidated (10). The rfb operon encoding this O3 antigen has been deposited in Genbank under accession number AB795941.1.

E. coli serotypes O8 and O9 have structurally the same O-specific mannose homopolysaccharide as Klebsiella serotypes O5 and O3, respectively. A monoclonal antibody that serotypically discriminates E. coli O9a, a subtype of E. coli O9, from E. coli O9 has been described (Sugiyama et al. 1998, J. Bacteriol. 180(10):2775-2778). E. coli O9 and O9a are structurally and serologically similar to each other.

The structure of E. coli O9 as well as the genetic determinants thereof is identical to those of the Klebsiella O3 antigen. A subtype of serogroup O9, i.e. E. coli O9a was proven to result from a point mutation within WbdA (7). The structure of O9a was shown to be a tetra-mannose structure (8).

TABLE
Structure of repeating units of O-specific mannose homopolysaccharide
(published in Sugiyama et al.)

| | Structure of the O-antigen |
|---|---|
| Serotype(s) | repeating unit |
| E. coli O8 and Klebsiella O5 | 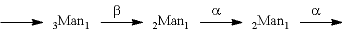 |
| E. coli O9a | 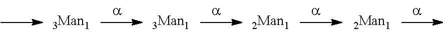 |
| E. coli O9 and Klebsiella O3 | 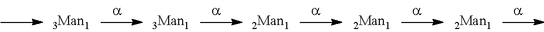 |

An anti-E. coli O9a monoclonal antibody has been described to cross-react with Klebsiella O3 polysaccharide, suggesting the presence of E. coli O9a type O polysaccharides in Klebsiella O3 strains (Kido et al. 1997, Microbiol. Immunol. 41:519-525. The antibody recognized the E. coli O9a polysaccharide but not the E. coli O9. The minimum number of mannose residues needed to define the O9 and O9a polysaccharide was determined to be four, and the 4-mannose structure has been described to be the shortest candidate for the epitope bound by the antibody.

Van der Meer et al. (Infection and Immunity 1994, 62(3):1052-1057) describe a monoclonal antibody (mAb) raised against Salmonella minnesota R595 and specific for a structure of the inner core, which is α-3-deoxy-D-manno-octulosonic acid. The antibody reacted with almost all O-serotypes of Klebsiella pneumoniae, suggesting an epitope in the core of the LPS like that in the inner core of S. minnesota.

WO2008/135446A2 discloses peptidic Klebsiella antigens and antibodies.

Pollack et al. (Journal of Clinical Investigation 1987, 79(5):1421-1430) describe mAbs recognizing epitopes in the core-Lipid A region of LPS.

Yokochi et al. (Infection and Immunity 1992, 60(11):4953-4956) describe adjuvant activity of LPS from K. pneumoniae. It is suggested that the adjuvanticity of Klebsiella O3 LPS might require a combination of the Klebsiella lipid A moiety and the mannose homopolysaccharide moiety.

Curvall et al. (Acta Chemica Scandinavica 1973, 27:2645-2649) disclose the structure of O-specific side chains in a Klebsiella O3 LPS. The Klebsiella O3:K58 LPS is described to be composed of pentasaccharide repeating units.

There is a need for new targets of Klebsiella pneumoniae. In particular, targets need to be identified which are immunorelevant and may be used for developing therapies and diagnostics.

### SUMMARY OF THE INVENTION

It is the objective of the present invention to provide for an antibody directed against K. pneumoniae with improved relevance to target the pathogen, to be used for the prevention or therapy of K. pneumoniae infections. It is further the objective to provide means and methods that are capable of diagnosing K. pneumoniae bacteria, in a rapid and reliable manner.

The object is solved by the subject of the present invention.

According to the invention, there is provided an isolated antibody that specifically recognizes an epitope of the lipopolysaccharide (LPS) O3b-antigen structure of Klebsiella pneumoniae, which is a O3b-epitope incorporated in O3b-antigen comprising the structure of Formula (I), including one or more O3b-antigen mannose homopolymer repeating units, wherein Formula (I) is:

MeP→3)-α-D-Manp-(1→2)-α-D-Manp-(1→3)-α-D-Manp-(1→[3)-α-D-Manp-(1→2)-α-D-Manp-(1→3)-α-D-Manp-(1→]ₙ

wherein

MeP is methyl phosphate; and

n is 0-50.

Specifically, the methyl phosphate group is situated at the non-reducing end of the mannose residue.

The O3b epitope is specifically characterized by the trimannose repeating unit set forth in Formula (I).

Specifically, the antibody is raised against the O3b-antigen structure, or obtained by engineering and selection techniques, and identified by binding to such structure or the O3b-epitope incorporated therein.

Specifically, the antibody is capable of binding such an O3b-epitope.

According to a specific aspect, the antibody cross-reacts with an O3a-epitope and/or an O3-epitope, wherein

a) the O3a-epitope is incorporated in the LPS O3a-antigen of Klebsiella pneumoniae comprising the structure of Formula (II), including one or more O3a-antigen mannose homopolymer repeating units, wherein Formula (II) is:

MeP→3)-α-D-Manp-(1→2)-α-D-Manp-(1→2)-α-D-Manp-(1→3)-α-D-Manp-(1→[3)-α-D-Manp-(1→2)-α-D-Manp-(1→2)-α-D-Manp-(1→3)-α-D-Manp-(1→]ₘ

wherein m is 0 to 50;

and

b) the O3-epitope is incorporated in the LPS O3-antigen of Klebsiella pneumoniae comprising the structure of Formula (III), including one or more O3-antigen mannose homopolymer repeating units, wherein Formula (III) is:

MeP→3)-α-D-Manp-(1→2)-α-D-Manp-(1→2)-α-D-Manp-(1→2)-α-D-Manp-(1→3)-α-D-Manp-(1→[3)-α-D-Manp-(1→2)-α-D-Manp-(1→2)-α-D-Manp-(1→2)-α-D-Manp-(1→3)-α-D-Manp-(1→]ₘ

wherein m is 0 to 50.

The O3a epitope is specifically characterized by the tetramannose repeating unit set forth in Formula (II).

The O3 epitope is specifically characterized by the pentamannose repeating unit set forth in Formula (III).

Such cross-reacting antibody is characterized by the O3-specificity directed to the O3b-epitope and further cross-specificity directed to one of or both of the O3a- and O3 epitopes.

Specifically, the antibody is a pan-O3 specific antibody, specifically recognizing or binding to the O3b-epitope and cross-reacting with the O3a-epitope and the O3-epitope. Cross-reactivity is specifically based on the presence of the O3b-epitope in the O3a and O3 LPS antigens.

For example, the antibody is a pan-O3 specific antibody described in the examples and designated 2F8-G6 or 4D3-A4, or a functional variant of such antibody e.g., a functional variant which substantially has the same binding specificity than the 2F8-G6 or 4D3-A4 antibody, or which competitively binds the O3b epitope. The 2F8-G6 is specifically characterized by the six CDR sequences and/or the VH/VL sequences incorporated in the deposited material referred to herein. The 4D3-A4 is specifically characterized by the six CDR sequences and/or the VH/VL sequences incorporated in the HC and LC sequences described herein.

Specifically, the 2F8-G6 or 4D3-A4 antibodies and functional variants thereof are characterized by the binding specificity directed to the O3b epitope and structure, which is also incorporated in the O3a and O3 structures, resulting in the cross-reactivity, also referred to as pan-O3 specificity.

For the purpose of providing variants, such antibodies are herein referred to as parent antibodies, and CDR or framework sequences are herein referred to as parent CDR or parent framework sequences.

According to a specific aspect, the variant antibody binds the same epitope as the parent antibody.

According to a further specific aspect, the variant antibody comprises the same binding site as the parent antibody.

Functionally active variant antibodies may differ in any of the VH or VL sequences, or share the common VH and VL sequences, and comprise modifications in the respective FR. The variant antibody derived from the parent antibody by mutagenesis may be produced by methods well-known in the art.

Functional variants of an antibody may specifically be engineered to obtain CDR mutated antibodies e.g., to improve the affinity of an antibody and/or to target the same epitope or epitopes near the epitope that is targeted by a parent antibody (epitope shift).

Specifically, the functionally active variant is a functionally active CDR variant which comprises at least one point mutation in the parent CDR sequence, and comprises or consists of the amino acid sequence that has at least 60% sequence identity with the parent CDR sequence, preferably at least 70%, at least 80%, at least 90% sequence identity.

Specific functional variants have an affinity to bind the O3b-antigen with a Kd of less than 10⁻⁸ M, preferably less than 10⁻⁹ M, preferably less than 10⁻¹⁰ M, preferably less than 10⁻¹¹ M e.g., with an affinity in the picomolar range.

A specific variant is e.g., a humanized variant of the parent antibody, wherein the parent CDR sequences are incorporated into human or humanized framework sequences, wherein optionally 1, 2, 3, or 4 amino acid residues of each of the parent CDR sequences may be further mutated by introducing point mutations to improve the stability, specificity and affinity of the parent or humanized antibody.

According to a specific aspect, the antibody comprises recombinant CDR and framework sequences e.g., of different origin, wherein at least one of the CDR and framework sequences includes human, humanized, chimeric, murine or affinity matured sequences, preferably wherein the framework sequences are of any immunoglobulin isotype, and in particular of an IgG antibody.

Variants of parent antibodies which are produced by affinity maturation, herein referred to as affinity-maturated variants, may have an increased binding affinity, with a Kd difference of at least 1 log, or 2 logs, or 3 logs, as compared to the parent antibody. Affinity maturated variants typically have an affinity to bind the O3b-antigen with a Kd of less than 10⁻⁸ M, or less than 10⁻⁹ M. If the parent antibody has an affinity with a Kd of less than 10⁻⁸ M, or less than 10⁻⁹ M, and the parent antibody is undergoing affinity maturation, the affinity matured variant may have an even higher affinity with a Kd of less than 10⁻⁹ M and less than 10⁻¹⁰ M, respectively.

According to a specific embodiment, the antibody is the 2F8-G6 antibody, or an antibody which competitively binds to its specific epitope, wherein the 2F8-G6 antibody is characterized by the heavy chain (VH) and light chain (VL) sequences incorporated in the deposited material as described herein.

Specifically, the 2F8-G6 antibody is characterized by

a) the VH incorporated in the deposited material DSM 32059; and

b) light VL incorporated in the deposited material DSM 32060.

According to another specific embodiment,

the antibody is the 4D3-A4 antibody or which competitively binds to its specific epitope.

Specifically, the 4D3-A4 antibody is characterized by any of

a) the 6 CDR sequences identified in FIG. 10, in particular by the CDR1, 2, 3, 4, 5, and 6 identified by SEQ ID 1, 2, 3, 4, 5, and 6, respectively, wherein numbering is according to Kabat; or by the CDR1, 2, 3, 4, 5, and 6 identified by SEQ ID 7, 8, 9, 10, 11, and 12, respectively, wherein numbering is according to IMGT; and/or

b) the VH and VL sequences identified in FIG. 10, in particular the VH sequence identified by SEQ ID 15 and the VL sequence identified by SEQ ID 16; and/or

c) the HC and LC sequences identified in FIG. 10, in particular the HC sequence identified by SEQ ID 13 and the LC sequence identified by SEQ ID 14.

Specifically, the CDR sequences according to Kabat as referred to herein are understood as those amino acid sequences of an antibody as determined according to Kabat nomenclature (see Kabat et al., Sequences of Proteins of Immunological Interest, 5^{th} Ed. Public Health Service, U.S. Department of Health and Human Services. (1991)).

Specifically, the CDR sequences according to IMGT as referred to herein are understood as those amino acid sequences of an antibody as determined according to the IMGT system (The international ImMunoGeneTics, Lefranc et al., 1999, Nucleic Acids Res. 27: 209-212).

Preferably, competition of binding is determined by competition ELISA analysis or by ForteBio analysis.

The antibody or the functional variant of any of the exemplified antibodies which competitively binds to any of the 2F8-G6 or 4D3-A4 antibodies is specifically characterized by a relative inhibition of binding to its target as determined by competition ELISA analysis or by ForteBio analysis, which relative inhibition is preferably greater than 30%.

According to a specific aspect, the antibody preferentially binds to the O3b-epitope relative to an O3a-epitope, or which does not cross-react with an O3a-epitope, wherein the O3a-epitope is incorporated in O3a-antigen repeating units of the LPS O3a-antigen structure of Klebsiella pneumoniae, wherein the O3a-antigen repeating unit is a mannose homopolymer of Formula (II).

According to a further specific aspect, the antibody preferentially binds to the O3b-epitope relative to an O3-epitope, or which does not cross-react with an O3-epitope, wherein the O3-epitope is incorporated in O3-antigen repeating units of the LPS O3-antigen structure of Klebsiella pneumoniae, wherein the O3-antigen repeating unit is a mannose homopolymer of Formula (III).

Specifically, the preferential binding is a characteristic feature of the antibody, which is capable of binding the O3b-epitope with a higher affinity and/or avidity than the other O3-antigen(s), in particular, the antibody preferentially binds to the O3b-epitope relative to a O3a-epitope and/or the O3-epitope e.g., with an affinity which is higher to bind the O3b-epitope or O3b-antigen as compared to any of the other O3-antigens. According to a specific embodiment, the antibody has at least two-fold greater affinity for binding the O3b-epitope or O3b-antigen as compared to any of the other O3-antigens, specifically with at least two-fold difference, or at least three-fold, at least four-fold, at least 5-fold, or even at least 10-fold difference e.g., difference in affinity and/or avidity. For example, the Kd difference to preferentially bind the O3b-antigen over the O3a-antigen and/or the O3-antigen is at least 0.5 or 1 log, or even at least 2 logs, or at least 3 logs different, as determined by an immunoassay, preferably immunoblotting, ELISA or other immunological methods.

According to a further specific aspect, the antibody does not cross-react with any of the O3a-epitope and the O3-epitope. Specifically, the antibody binds to any of the other O3-antigen(s) with a lower affinity e.g., where the Kd difference to preferentially bind the O3b-epitope or O3b-antigen over any of the other O3-antigen(s) is at least 2 logs, preferably at least 3 logs.

According to a further specific aspect, the antibody does not cross-react with an epitope of non-O3 LPS molecules of Klebsiella pneumoniae. Such non-O3 LPS molecules are e.g., O1, O2, O4, O12 LPS molecules. Specifically, the antibody does not cross-react with any other K. pneumoniae antigen, and/or the antibody binds to any other K. pneumoniae antigen with a lower affinity e.g., where the Kd difference to preferentially bind the O3b-epitope or O3b-antigen over other K. pneumoniae antigens (other than the O3b-antigen) is at least 2 logs, preferably at least 3 logs.

Specifically, the non-cross-reaction is determined by an ELISA assay or immunoblot using the O3b-antigen and the further antigen(s), to which the antibody does not significantly bind.

According to a specific embodiment, the antibody has an affinity to bind the O3b-epitope with a Kd of less than 10⁻⁷ M, preferably less than 10⁻⁸ M, even more preferably less than 10⁻⁹ M, or preferably less than 10⁻¹⁰ M, or preferably less than 10⁻¹¹M e.g., with an affinity in the picomolar range.

Specifically, the pan-O3 specific antibody is capable of binding each of the O3b-epitope, the O3a-epitope and the O3-epitope with a high affinity, such as with a Kd of less than 10⁻⁷ M, preferably less than 10⁻⁸ M, even more preferably less than 10⁻⁹ M.

According to a specific aspect, the antibody is a neutralizing antibody. Specifically, the antibody is neutralizing endotoxin of Klebsiella pneumoniae strains expressing O3b or any of O3a or O3 LPS molecules e.g., as determined by an in vitro or in vivo detection method. Specifically, the antibody neutralizes the endotoxic effect of specific LPS molecules in vitro.

Specifically, the antibody is neutralizing endotoxin of Klebsiella pneumoniae strains expressing the O3b-epitope or O3b-antigen, wherein the neutralization potency is at least the potency of a reference antibody (e.g., the exemplary antibody described herein designated 4D3-A4).

Specifically, the antibody is a cross-neutralizing antibody, which has a neutralization potency to neutralize endotoxin of Klebsiella pneumoniae strains serotype O3b, and at least one of or both of O3a, and O3, which neutralization potency is at least the potency of a reference antibody (e.g., the exemplary antibody described herein designated 4D3-A4).

Specifically, the antibody is provided for bactericidal killing of Klebsiella pneumoniae of the O3b-type.

According to a specific aspect, immunotherapy using the antibody of the invention may effectively protect against live bacterial challenge e.g., as determined in various animal models.

The antibody may specifically neutralize lethal endotoxemia. Such functional activity may be determined in an appropriate in vivo model (challenge with purified LPS).

The antibody is specifically effective against Klebsiella pneumoniae of the O3b-type by complement-mediated killing e.g., as determined by an in vitro serum bactericidal assay (SBA) e.g., with at least 20% killing of bacteria above the control samples (no antibody or irrelevant control mAb added).

The antibody is specifically effective against Klebsiella pneumoniae of the O3b-type by antibody mediated phagocytosis e.g., as determined by an in vitro opsonophagocytotic killing assay (OPK) e.g., with at least 20% uptake of input bacteria or 20% lower end CFU count above the control samples (no antibody or irrelevant control mAb added).

The antibody is specifically effective against Klebsiella pneumoniae of the O3b-type by neutralizing endotoxin functions e.g., as determined by an in vitro LAL assay, or toll-like receptor 4 (TLR4) reporter assay e.g., with at least 20% reduction in endotoxin activities in comparison to control samples (no antibody or irrelevant control mAb added).

According to a further specific aspect, the antibody neutralizes the targeted pathogen in animals, including both, human and non-human animals, and inhibits pathogenesis in vivo, preferably any models of primary and secondary bacteremia, pneumonia, urinary tract infection, liver abscess, peritonitis, or meningitis.

Specifically, the antibody is a full-length monoclonal antibody, an antibody fragment thereof comprising at least one antibody domain incorporating the binding site, or a fusion protein comprising at least one antibody domain incorporating the binding site, specifically wherein the antibody is a non-naturally occurring antibody which comprises a randomized or artificial amino acid sequence. Specifically, the antibody is any of full-length IgG1, a bispecific IgG1, or a F(ab′)₂-fragment.

Specifically, the antibody is an antibody selected from the group consisting of murine, lama, rabbit, goat, cow, chimeric, humanized or human antibodies, heavy-chain antibodies, Fab, Fd, scFv and single-domain antibodies like VH, VHH or VL, preferably a human IgG antibody or a murine IgG antibody.

According to a specific embodiment, the antibody comprises at least an antibody heavy chain variable region or domain (VH), which is e.g., characterized by any of the VH-CDR1 to VH-CDR3 sequences of the 2F8-G6 or 4D3-A4 antibody, or functionally active CDR variants thereof. CDR sequences are in particular designated according to the numbering system of Kabat.

According to a specific aspect, the antibody only comprises a VH domain as antigen binding moiety, thus, may comprise VH-CDR1-3 e.g., of the 2F8-G6 or 4D3-A4 antibody or a functional CDR variant of the 2F8-G6 or 4D3-A4 antibody, without a respective VL domain.

According to another specific aspect, the antibody comprises a VH domain and further comprises an antibody light chain variable region or domain (VL), which is e.g., characterized by any of the VL-CDR1 to VL-CDR3 sequences of the 2F8-G6 or 4D3-A4 antibody, or functionally active CDR variants thereof.

According to another specific aspect, the antibody comprises the binding site of any one of the 2F8-G6 or 4D3-A4 antibodies, characterized by 6 CDR sequences which are the VH-CDR1-3 and the VL-CDR1-3 sequences, or a functionally active CDR variant thereof.

CDR sequences are in particular designated according to the numbering system of Kabat.

According to a specific aspect, the invention provides for the exemplary (parent) 2F8-G6 or 4D3-A4 antibody to produce antibody variants of such parent antibody, in particular including variants binding to essentially the same epitope, as the parent antibody which is characterized by the specific binding site formed by either the VH sequence alone or both, the VH and the VL amino acid sequences as identified in the sequence listing, or obtainable from the deposited material, or else by the respective CDR sequences. Such antibodies may e.g., be functionally active variant antibodies obtained by modifying the respective CDR or antibody sequence of the parent antibody. It is well understood that any antibody sequence as described herein is considered a “parent” sequence which is subject to variation e.g., by point mutations.

The 2F8-G6 and 4D3-A4 antibodies described in the examples are of murine origin, which have been chimerized with human sequences. Variants which are obtained by humanization and optionally affinity maturation may be engineered using well-known techniques. These variant antibodies bind to the target antigen, thus, are considered functionally active. It is feasible that also variant VH or VL domains e.g., with modifications in the respective FR or CDR sequences may be used, which are functionally active e.g., binding to the same epitope or comprising the same binding site or having the same binding characteristics as the parent antibody. It is also feasible that some of the FR or CDR sequences of the antibodies described herein may be exchanged by those of other antibodies.

Specifically the antibody comprises a functionally active CDR variant of any of the CDR sequences of the 2F8-G6 or 4D3-A4 antibody, wherein the functionally active CDR variant comprises at least one of

a) 1, 2, or 3 point mutations in the parent CDR sequence; and/or

b) 1 or 2 point mutations in any of the four C-terminal or four N-terminal, or four centric amino acid positions of the parent CDR sequence; and/or

c) at least 60% sequence identity with the parent CDR sequence;

preferably wherein the functionally active CDR variant comprises 1 or 2 point mutations in any CDR sequence consisting of less than 4 or 5 amino acids.

Specifically, the functionally active variant differs from the parent antibody in at least one point mutation in the amino acid sequence, preferably in the CDR, wherein the number of point mutations in each of the CDR amino acid sequences is either 0, 1, 2 or 3.

According to a specific aspect, the point mutation is any of an amino acid substitution, deletion and/or insertion of one or more amino acids.

Specifically, the antibody is of human, humanized, chimeric, or murine origin.

According to a specific aspect, the antibody of the invention comprises CDR and framework sequences, wherein at least one of the CDR and framework sequences includes human, humanized, chimeric, murine or affinity matured sequences, preferably wherein the framework sequences are of an IgG antibody e.g., of an IgG1, IgG2, IgG3, or IgG4 subtype, or of an IgA1, IgA2, IgD, IgE, or IgM antibody.

Specific antibodies are provided as framework mutated antibodies e.g., to improve manufacturability or tolerability of a parent antibody e.g., to provide an improved (mutated) antibody which has a low immunogenic potential, such as humanized antibodies with mutations in any of the CDR sequences and/or framework sequences as compared to a parent antibody.

Specifically, the antibody is a monoclonal antibody. In particular, the antibody is a non-naturally occurring antibody, such as including artificial variable and/or constant domain sequences e.g., sequences (such as CDR of Fc sequences) obtained from a library of randomized sequences.

According to a specific aspect, the antibody is provided for use in treating a subject at risk of or suffering from Klebsiella pneumoniae infection or colonization comprising administering to the subject an effective amount of the antibody to limit the infection in the subject or to ameliorate a disease condition resulting from said infection, preferably for treatment or prophylaxis of any of primary and secondary bacteremia, pneumonia, urinary tract infection, liver abscess, peritonitis, or meningitis.

Preferably, the antibody used for therapeutic purposes is a neutralizing antibody neutralizing LPS of the target pathogen.

Therefore, the invention further provides for a method of treating a subject by administering an effective amount of the antibody in the respective indications.

Specifically, the antibody is used by administering to the subject an effective amount of the antibody to limit the infection in the subject or to ameliorate a disease condition resulting from said infection, preferably for treatment or prophylaxis of any of primary and secondary bacteremia, pneumonia, urinary tract infection, liver abscess, peritonitis, or meningitis.

Specifically, the subject is a human being. Specifically, the subject is any human being who is healthy or suffering from a disease. Specifically, the human being is an immunocompromised or immunosuppressed patient, or a contact thereof.

The invention further provides for a pharmaceutical preparation comprising the antibody as described herein, preferably comprising a parenteral (e.g., i.v. or i.m.) or mucosal (e.g., oral) formulation, optionally containing a pharmaceutically acceptable carrier or excipient. A mucosal formulation is e.g., emulsified, nanoparticulated, or nebulized.

Such pharmaceutical composition may contain the antibody as the sole active substance, or in combination with other active substances, or a cocktail of active substances, such as a combination or cocktail of at least two or three different antibodies.

According to the invention, the antibody of the invention is specifically provided for medical, diagnostic or analytical use.

The invention further provides for the use of the antibody described herein for diagnosis of Klebsiella pneumoniae infection or colonization, or an associated disease such as primary and secondary bacteremia, pneumonia, urinary tract infection, liver abscess, peritonitis, or meningitis in a subject.

Specifically, the antibody is provided for use as described herein, wherein a systemic infection or colonization with Klebsiella pneumoniae of the O3b-type in a subject is determined ex vivo by contacting a biological sample of said subject with the antibody, wherein a specific immune reaction of the antibody determines the infection or colonization.

Specifically, the biological samples is a body fluid or tissue sample, preferably a sample selected from the group consisting of a blood sample, stool sample, skin sample, urine sample, cerebrospinal fluid, and a respiratory tract specimen such as endotracheal aspirates, pleural fluid, lung tap, nasal swab or sputum, or a Klebsiella pneumoniae isolate originating from any of the foregoing. Specifically, a sample of body fluid is tested for the specific immune reaction, which sample is selected from the group consisting of urine, blood, blood isolates or blood culture, aspirate, sputum, lavage fluid of intubated subjects and stool.

Specifically, the biological sample is treated to produce a Klebsiella pneumoniae isolate originating from the biological sample, which isolate may be further characterized for its O3b genotype or phenotype, and/or the level of O3b antigen expression. Preferable sample preparation methods for producing bacterial isolates are employing bacterial enrichment and cultivation steps.

Specifically, the biological sample is treated to determine the O3b-antigen level directly in the sample, optionally following preparatory steps of enrichment or purification to reduce matrix effects and to increase the specificity and sensitivity of the test. Preparatory steps include culturing of the biological specimen according to standard culture procedures such as but not exclusively being hemocultures in standard growth media as well as the culturing of specimens on solid agar (including phenotyping—i.e. antibiogram) as performed in routine microbiology laboratories. Bacteria may be sub-cultured for expansion of CFU in different growth media (standard media and/or chemically defined media; high nutrient, low nutrient, limited growth media composition) to enhance expression of virulence factors. Bacterial suspensions may be prepared and washed in standard buffer solutions to remove potential matrix effects.

Specifically, the O3b-antigen is determined by at least one of an immunoassay, preferably any of ELISA, CIA, RIA, IRMA, agglutination assay, immunochromatography, dipstick assay and Western-blot/immunoblot, biosensors, array technology, or mass-spectrometry, nuclear magnetic resonance (NMR).

Specifically, the diagnostic use according to the invention refers to determining the serotype of Klebsiella pneumoniae in vitro from a pure Klebsiella pneumoniae culture recovered from a clinical specimen, to determine whether the bacterium is of the O3b-type, or not.

The invention further provides for a diagnostic preparation of the antibody described herein, comprising the antibody and a further diagnostic reagent in a composition or a kit of parts, comprising the components
a) the antibody as described herein; and
b) the further diagnostic reagent;
c) and optionally a solid phase to immobilize at least one of the antibody and the diagnostic reagent.

The diagnostic preparation optionally comprises the antibody of the invention and the further diagnostic reagent in a composition or a kit of parts.

The diagnostic kit preferably comprises all essential components to determine the O3b-antigen expression in the biological sample, optionally without common or unspecific substances or components, such as water, buffer or excipients. The storage stable kit can be stored preferably at least 6 months, more preferably at least 1 or 2 years. It may be composed of dry (e.g., lyophilized) components, and/or include preservatives.

The preferred diagnostic kit is provided as a packaged or prepackaged unit e.g., wherein the components are contained in only one package, which facilitates routine experiments. Such package may include the reagents necessary for one or more tests e.g., suitable to perform the tests of a series of biological samples. The kit may further suitably contain a O3b-antigen preparation as a standard or reference control.

The diagnostic composition may be a reagent ready-to-use in a reaction mixture with the biological sample, or a conserved form of such reagent e.g., a storage-stable form such as lyophilized; snap-frozen (e.g., in liquid nitrogen), ultra-low-temperature storage (−70° C. and −80° C.), cold-storage (−20° C. and 5° C.) and controlled room temperature (15° C.−27° C.); standard sample storage as e.g., glycerol-stocks, tissue paraffin-blocks, (buccal) swabs and other standard biological sample storage methods, which conserved form of a reagent can be reconstituted or prepared to obtain a ready-to-use reagent. Such ready-to-use reagent is typically in the form of an aqueous solution, specifically (physiological) buffer conditions (e.g., EDTA buffered, phosphate buffer, HBSS, citrate buffer etc.).

Specifically, the further diagnostic reagent is a reagent specifically reacting with the antibody and/or the reaction product of the antibody binding to its antigen. An appropriate diagnostic reagent is suitably used for performing an immunoassay for diagnosing or monitoring, in a subject, the Klebsiella pneumoniae infection or colonization. The appropriate diagnostic reagent can be a solvent, a buffer, a dye, an anticoagulant, a ligand that specifically binds to the antibody of the invention and/or the antibody-antigen immune complex.

Specifically, the invention provides for a diagnostic preparation of an antibody of the invention, optionally containing the antibody with a label and/or a further diagnostic reagent with a label, such as a reagent specifically recognizing the antibody or an immune complex of the antibody with the respective target antigen, and/or a solid phase to immobilize at least one of the antibody and the diagnostic reagent.

Specifically, the further diagnostic reagent is a diagnostic label or a reagent specifically reacting with the antibody and/or the reaction product of the antibody binding to its antigen.

The antibody or the diagnostic reagent can be directly labeled or indirectly labeled. The indirect label may comprise a labeled binding agent that forms a complex with the antibody or diagnostic reagent to the O3b-antigen.

The label is typically a molecule or part of a molecule that can be detected in an assay. Exemplary labels are chromophores, fluorochromes, or radioactive molecules. In some embodiments the antibody or diagnostic reagent is conjugated to a detectable label which may include molecules that are themselves detectable (e.g., fluorescent moieties, electrochemical labels, metal chelates, etc.) as well as molecules that may be indirectly detected by production of a detectable reaction product (e.g., enzymes such as horseradish peroxidase, alkaline phosphatase, etc.) or by a specific binding molecule which itself may be detectable (e.g., biotin, digoxigenin, maltose, oligohistidine, 2,4-dintrobenzene, phenylarsenate, ssDNA, dsDNA, etc.).

Preferred diagnostic preparations or assays comprise the antibody of the invention immobilized on a solid phase e.g., latex beads, gold particles, etc. e.g., to test agglutination by the antibody of bacteria of the O3b-type obtained from a sample to be tested.

The invention further provides for a method of diagnosing Klebsiella pneumoniae infection or colonization in a subject caused by a Klebsiella pneumoniae strain, comprising
a) providing the antibody as described herein; and
b) detecting if the antibody specifically immunoreacts with the O3b-epitope in a biological sample of the subject to be tested, thereby diagnosing Klebsiella pneumoniae infection or colonization.

Specifically, the biological sample is a body fluid or tissue sample, preferably a sample selected from the group consisting of a blood sample, stool sample, skin sample, urine sample, cerebrospinal fluid, and a respiratory tract specimen such as endotracheal aspirates, pleural fluid, lung tap, nasal swab or sputum, or a Klebsiella pneumoniae isolate originating from any of the foregoing.

Such diagnosis is specifically indicated in case of a MDR Klebsiella pneumoniae infection of colonization, in particular addressing MDR Klebsiella pneumoniae of the O3b-type. Optionally, a diagnostic assay may involve two different antibodies with different specificity and/or affinity to bind O3b-antigen and/or any of the further O3a-antigen and O3-antigen, so to possibly differentiate between the various O3-antigens.

According to a specific aspect, the invention provides for companion diagnostics to determine the infection of a subject with Klebsiella pneumoniae by the diagnostics of the invention or the diagnostic method of the invention, to provide for the basis of treatment with a therapeutic against such infection e.g., employing immunotherapy, such as treating with an antibody of the invention.

According to a specific aspect, the invention provides for a sensitive bedside diagnostics to diagnose infection of a subject with Klebsiella pneumoniae by determining free LPS e.g., from clinical specimen where the amount of live bacteria is limited. The sensitivity of such assay is specifically less than 100 ng preferably less than 10 ng of LPS.

The invention further provides for an isolated nucleic acid encoding the antibody as described herein.

The invention further provides for an expression cassette or a plasmid comprising a coding sequence to express a proteinaceous construct, such as comprising or consisting of a polypeptide or protein, or a protein derivative, comprising a VH and/or VL of the antibody as described herein.

The invention further provides for a host cell comprising an expression cassette or a plasmid as described herein.

The invention further provides for a method of producing the antibody as described herein, wherein the host cell is cultivated or maintained under conditions to produce said antibody.

Specifically preferred is a host cell and a production method employing such host cell, which host cell comprises
the plasmid or expression cassette of the invention, which incorporates a coding sequence to express the antibody light chain; and
the plasmid or expression cassette of the invention, which incorporates a coding sequence to express the antibody heavy chain.

According to a further aspect, the invention provides for a method of producing an antibody of the invention, comprising
a) immunizing a non-human animal with the O3b-antigen of Klebsiella pneumoniae and isolating B-cells producing antibodies;
b) forming immortalized cell lines from the isolated B-cells;
c) screening the cell lines to identify a cell line producing a monoclonal antibody that specifically binds to the O3b-antigen and optionally the O3a-antigen and/or the O3-antigen e.g., wherein preferential binding to O3b-antigen as compared to any of the other O3-antigen(s) is determined; and
d) producing the monoclonal antibody, or a humanized or human form of the antibody, or a derivative thereof with the same epitope binding specificity as the monoclonal antibody.

Alternatively, the method comprises as a screening step: screening the B-cells of human donors isolated from peripheral blood and cloning and sequencing of immunoglobulin genes following single-cell sorting of B-cells binding to target antigens.

The invention further provides for a method of identifying a candidate antibody comprising:
a) providing a sample containing an antibody or antibody-producing cell; and
b) assessing for binding of an antibody in or produced by the sample with a O3b-epitope as defined herein, wherein a positive reaction between the antibody and the epitope identifies the antibody as candidate antibody.

The invention further provides for a method of identifying a candidate antibody comprising:
a) providing a sample containing an antibody or antibody-producing cell; and
b) assessing for binding of an antibody in or produced by the sample with the O3b-epitope as defined herein, wherein a specific positive reaction between the antibody and the O3b-epitope relative to the O3a-epitope and/or O3-epitope as defined herein, or relative to any non-O3 LPS molecule of Klebsiella pneumoniae identifies the antibody as candidate antibody.

Specifically, the pan-O3 specific antibody is identified, if the candidate antibody recognizes the O3b-epitope, the O3a-epitope and the O3-epitope.

Specifically, the pan-O3 specific antibody is capable of binding each of the O3b-epitope, the O3a-epitope and the O3-epitope with a high affinity, such as with a Kd of less than 10⁻⁷ M, preferably less than 10⁻⁸ M, even more preferably less than 10⁻⁹ M.

Specifically, the pan-O3 specific antibody does not cross-react or does not significantly cross-react with non-O3 epitopes, such as O1, O2, O4 and O12.

The invention further provides for a method of producing the antibody as described herein, comprising

a) providing the candidate antibody identified as described herein; and

b) producing a monoclonal antibody, or a humanized or human form of the candidate antibody, or a derivative thereof with the same epitope binding specificity as the candidate antibody.

### FIGURES

FIG. 1. Structure of the classical O3 antigen of Klebsiella pneumoniae. The disaccharide structure formed by N and Q is the so-called adaptor bridging the penta-mannose O-antigen subunits to the carrier-primer (CP). The last O-antigen repeat is capped by a terminator molecule (T), which in fact is a 3-linked methyl-phosphate as elucidated by Kubler-Kielb et al. (4)

FIG. 2. Heterogeneity of the LPS patterns of K. pneumoniae strains belonging to the O3 serogroup. Lanes 1: PCM-11, 2:Kp14, 3:Kp62, 4: Kp18, 5: Kp35

FIG. 3. ProQ staining (A) and immunoblot (B and C) of LPS samples purified from different O3 (lanes 4-9) and unrelated serotypes (lanes 1-3) strains of K. pneumoniae. Immunoblots were performed with 1 μg/ml of mAbs 1G6-B8 (B) or 2F8-G6 (C). Lanes 1: #63 (O1:K2), 2:#79 (O2:K27), 3: Kp108 (O5), 4: PCM-11, 5: Kp14, 6: Kp62, 7: Kp18, 8: Kp35, 9: Kp82.

FIG. 4. ¹H NMR (left panels) and MALDI-TOF MS (right panels) spectra of O-PS fractions 1a, 1b, 1c of K. pneumoniae Kp81 LPS. The inset structure present Kp81 O-PS.*MeP—methyl phosphate.

FIG. 5. Negative ion-mode MALDI-TOF mass spectrum of fraction 1b of K. pneumoniae Kp81 LPS.

FIG. 6. Surface staining of live Klebsiella strains expressing different O3 antigens by mAbs of various specificities.

FIG. 7. Table 1. ¹H and ¹³C NMR chemical shifts and inter-residue connectivities observed for O-PS (1a) isolated from K. pneumoniae LPS, strain Kp81. MeP: 3.61, 3.63/53.7, J_{P,H} of 11.0 Hz. ³¹P, ¹H HMBC showed correlation between P (˜2 ppm) and protons of MeP (3.61 and 3.63 ppm) and H-3 of C′ (4.26 ppm). ³¹P, ¹H HMQC-TOCSY showed correlation between P and H-1, H-2, H-3, H-4,H-5 of C′ residue. Anomeric configuration (α) of sugar residues was determined on the basis of J_{H1,C1} of 171-174 Hz. ^{b, c, d, e, f, g}-overlapped signals.

FIG. 8. Serum susceptibility of Klebsiella pneumoniae O3 strains. Mid-log cultures (3×10³ CFU/ml) of the indicated O3 subtypes were incubated in 50% normal human serum. Bacterial numbers were determined at timepoints 0, 90, and 180 minutes in duplicates.

FIG. 9. In vitro neutralization of TLR-4 signaling of LPS extracted from an O3a (A) or O3b (B) Klebsiella pneumoniae strain. LPS was mixed and incubated in a 96 well plate with different concentrations of antibodies or polymyxin B for 30 minutes at room temperature. Afterwards, 5×10⁴ hTLR4 HEK Blue cells (InvivoGen) per well were added to the reaction and the mixtures were incubated overnight at 37° C. and 5% CO₂. Absorbance at OD630 was measured and the neutralization capacity was expressed as % inhibition of secreted alkaline phosphatase (SEAP) induction with the formula: % Inhibition=100−[100×SEAP (mAb)/SEAP (LPS only control)], where SEAP is the signal of each treatment relative to the signal of the mock non-treated control in absence of LPS.

FIG. 10. 4D3-A4 antibody sequences

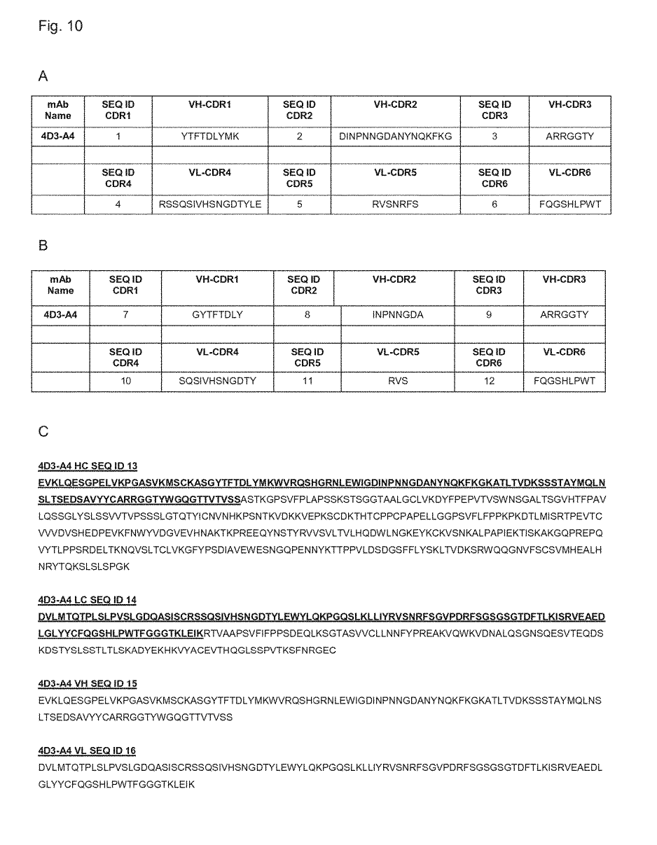

CDR sequences of antibody 4D3-A4 identified according to the Kabat system, CDR1 (SEQ ID 1), CDR2 (SEQ ID 2), CDR3 (SEQ ID 3), CDR4 (SEQ ID 4), CDR5 (SEQ ID 5), CDR6 (SEQ ID 6), (A); and listing the same antibody designating the CDR region according to the IMGT system, CDR1 (SEQ ID 7), CDR2 (SEQ ID 8), CDR3 (SEQ ID 9), CDR4 (SEQ ID 10), CDR5 (SEQ ID 11), CDR6 (SEQ ID 12), (B).

### Abbreviations

CDR1=VH-CDR1

CDR2=VH-CDR2

CDR3=VH-CDR3

CDR4=VL-CDR4 or VL-CDR1

CDR5=VL-CDR5 or VL-CDR2

CDR6=VL-CDR6 or VL-CDR3

Full length sequences of heavy chain (HC, SEQ ID 13) and light chain (LC, SEQ ID 14) identifying the variable and constant regions VH (SEQ ID 15) and VL (SEQ ID 16), (C).

Variable region VH or VL: BOLD AND UNDERLINED CAPS LETTERS

Constant region: NORMAL CAPS LETTERS

### DETAILED DESCRIPTION

The term “antibody” as used herein shall refer to polypeptides or proteins that consist of or comprise antibody domains, which are understood as constant and/or variable domains of the heavy and/or light chains of immunoglobulins, with or without a linker sequence. Polypeptides are understood as antibody domains, if comprising a beta-barrel structure consisting of at least two beta-strands of an antibody domain structure connected by a loop sequence. Antibody domains may be of native structure or modified by mutagenesis or derivatization e.g., to modify the antigen binding properties or any other property, such as stability or functional properties, such as binding to the Fc receptors FcRn and/or Fc gamma receptor.

The antibody as used herein has a specific binding site to bind one or more antigens or one or more epitopes of such antigens, specifically comprising a CDR binding site of a single variable antibody domain, such as VH, VL or VHH, or a binding site of pairs of variable antibody domains, such as a VL/VH pair, an antibody comprising a VL/VH domain pair and constant antibody domains, such as Fab, F(ab′), (Fab)₂, scFv, Fv, or a full length antibody.

The term “antibody” as used herein shall particularly refer to antibody formats comprising or consisting of single variable antibody domain, such as VH, VL or VHH, or combinations of variable and/or constant antibody domains with or without a linking sequence or hinge region, including pairs of variable antibody domains, such as a VL/VH pair, an antibody comprising or consisting of a VL/VH domain pair and constant antibody domains, such as heavy-chain antibodies, Fab, F(ab′), (Fab)₂, scFv, Fd, Fv, or a full-length antibody e.g., of an IgG type (e.g., an IgG1, IgG2, IgG3, or IgG4 subtype), IgA1, IgA2, IgD, IgE, or IgM antibody. The term “full length antibody” can be used to refer to any antibody molecule comprising at least most of the Fc domain and other domains commonly found in a naturally occurring antibody monomer. This phrase is used herein to emphasize that a particular antibody molecule is not an antibody fragment.

The term “antibody” shall specifically include antibodies in the isolated form, which are substantially free of other antibodies directed against different target antigens or comprising a different structural arrangement of antibody domains. Still, an isolated antibody may be comprised in a combination preparation, containing a combination of the isolated antibody e.g., with at least one other antibody, such as monoclonal antibodies or antibody fragments having different specificities.

The term “antibody” shall apply to antibodies of animal origin, including human species, such as mammalian, including human, murine, rabbit, goat, lama, cow and horse, or avian, such as hen, which term shall particularly include recombinant antibodies which are based on a sequence of animal origin e.g., human sequences.

The term “antibody” further applies to chimeric antibodies with sequences of origin of different species, such as sequences of murine and human origin.

The term “chimeric” as used with respect to an antibody refers to those antibodies wherein one portion of each of the amino acid sequences of heavy and light chains is homologous to corresponding sequences in antibodies derived from a particular species or belonging to a particular class, while the remaining segment of the chain is homologous to corresponding sequences in another species or class. Typically the variable region of both light and heavy chains mimics the variable regions of antibodies derived from one species of mammals, while the constant portions are homologous to sequences of antibodies derived from another. For example, the variable region can be derived from presently known sources using readily available B-cells or hybridomas from non-human host organisms in combination with constant regions derived from, for example, human cell preparations.

The term “antibody” may further apply to humanized antibodies.

The term “humanized” as used with respect to an antibody refers to a molecule having an antigen binding site that is substantially derived from an immunoglobulin from a non-human species, wherein the remaining immunoglobulin structure of the molecule is based upon the structure and/or sequence of a human immunoglobulin. The antigen binding site may either comprise complete variable domains fused onto constant domains or only the complementarity determining regions (CDR) grafted onto appropriate framework regions in the variable domains. Antigen-binding sites may be wild-type or modified e.g., by one or more amino acid substitutions, preferably modified to resemble human immunoglobulins more closely. Some forms of humanized antibodies preserve all CDR sequences (for example a humanized mouse antibody which contains all six CDRs from the mouse antibody). Other forms have one or more CDRs which are altered with respect to the original antibody.

The term “antibody” further applies to human antibodies.

The term “human” as used with respect to an antibody, is understood to include antibodies having variable and constant regions derived from human germline immunoglobulin sequences. The human antibody of the invention may include amino acid residues not encoded by human germline immunoglobulin sequences (e.g., mutations introduced by random or site-specific mutagenesis in vitro or by somatic mutation in vivo), for example in the CDRs. Human antibodies include antibodies isolated from human immunoglobulin libraries or from animals transgenic for one or more human immunoglobulin.

The term “antibody” specifically applies to antibodies of any class or subclass. Depending on the amino acid sequence of the constant domain of their heavy chains, antibodies can be assigned to the major classes of antibodies IgA, IgD, IgE, IgG, and IgM, and several of these may be further divided into subclasses (isotypes), e.g., IgG1, IgG2, IgG3, IgG4, IgA1, and IgA2.

The term further applies to monoclonal or polyclonal antibodies, specifically a recombinant antibody, which term includes all antibodies and antibody structures that are prepared, expressed, created or isolated by recombinant means, such as antibodies originating from animals e.g., mammalians including human, that comprises genes or sequences from different origin e.g., murine, chimeric, humanized antibodies, or hybridoma derived antibodies. Further examples refer to antibodies isolated from a host cell transformed to express the antibody, or antibodies isolated from a recombinant, combinatorial library of antibodies or antibody domains, or antibodies prepared, expressed, created or isolated by any other means that involve splicing of antibody gene sequences to other DNA sequences.

It is understood that the term “antibody” also refers to derivatives of an antibody, in particular functionally active derivatives. An antibody derivative is understood as any combination of one or more antibody domains or antibodies and/or a fusion protein, in which any domain of the antibody may be fused at any position of one or more other proteins, such as other antibodies e.g., a binding structure comprising CDR loops, a receptor polypeptide, but also ligands, scaffold proteins, enzymes, toxins and the like. A derivative of the antibody may be obtained by association or binding to other substances by various chemical techniques such as covalent coupling, electrostatic interaction, di-sulphide bonding etc. The other substances bound to the antibody may be lipids, carbohydrates, nucleic acids, organic and inorganic molecules or any combination thereof (e.g., PEG, prodrugs or drugs). In a specific embodiment, the antibody is a derivative comprising an additional tag allowing specific interaction with a biologically acceptable compound. There is not a specific limitation with respect to the tag usable in the present invention, as far as it has no or tolerable negative impact on the binding of the antibody to its target. Examples of suitable tags include His-tag, Myc-tag, FLAG-tag, Strep-tag, Calmodulin-tag, GST-tag, MBP-tag, and S-tag. In another specific embodiment, the antibody is a derivative comprising a label. The term “label” as used herein refers to a detectable compound or composition which is conjugated directly or indirectly to the antibody so as to generate a “labeled” antibody. The label may be detectable by itself e.g., radioisotope labels or fluorescent labels, or, in the case of an enzymatic label, may catalyze chemical alteration of a substrate compound or composition which is detectable.

The preferred derivatives as described herein are functionally active with regard to the antigen binding, preferably which have a potency to combat K. pneumoniae e.g., as determined in an SBA, OPK or LAL assay, or to protect against bacterial challenge or to neutralize endotoxemia.

Specifically, an antibody derived from an antibody of the invention may comprise at least one or more of the CDR regions or CDR variants thereof being functionally active in differentially binding to the O3b-antigen e.g., specifically or selectively binding the O3b-antigen.

Antibodies derived from a parent antibody or antibody sequence, such as a parent CDR or FR sequence, are herein particularly understood as mutants or variants obtained by e.g., in silico or recombinant engineering or else by chemical derivatization or synthesis.

It is understood that the term “antibody” also refers to variants of an antibody, including antibodies with functionally active CDR variants of a parent CDR sequence, and functionally active variant antibodies of a parent antibody.

Specifically, an antibody derived from an antibody of the invention may comprise at least one or more of the CDR regions or CDR variants thereof e.g., at least 3 CDRs of the heavy chain variable region and/or at least 3 CDRs of the light chain variable region, with at least one point mutation in at least one of the CDR or FR regions, or in the constant region of the HC or LC, being functionally active e.g., specifically binding the O3b-antigen.

The term “variant” shall particularly refer to antibodies, such as mutant antibodies or fragments of antibodies e.g., obtained by mutagenesis methods, in particular to delete, exchange, introduce inserts into a specific antibody amino acid sequence or region or chemically derivatize an amino acid sequence e.g., in the constant domains to engineer the antibody stability, effector function or half-life, or in the variable domains to improve antigen-binding properties e.g., by affinity maturation techniques available in the art. Any of the known mutagenesis methods may be employed, including point mutations at desired positions e.g., obtained by randomization techniques. In some cases positions are chosen randomly e.g., with either any of the possible amino acids or a selection of preferred amino acids to randomize the antibody sequences. The term “mutagenesis” refers to any art recognized technique for altering a polynucleotide or polypeptide sequence. Preferred types of mutagenesis include error prone PCR mutagenesis, saturation mutagenesis, or other site directed mutagenesis.

The term “variant” shall specifically encompass functionally active variants.

The term “functionally active variant” of a CDR sequence as used herein, is understood as a “functionally active CDR variant”, and the “functionally active variant” of an antibody as used herein, is understood as “functionally active antibody variant”. The functionally active variant means a sequence resulting from modification of this sequence (a parent antibody or a parent sequence) by insertion, deletion or substitution of one or more amino acids, or chemical derivatization of one or more amino acid residues in the amino acid sequence, or nucleotides within the nucleotide sequence, or at either or both of the distal ends of the sequence e.g., in a CDR sequence the N-terminal and/or C-terminal 1, 2, 3, or 4 amino acids, and/or the centric 1, 2, 3, or 4 amino acids (i.e. in the midst of the CDR sequence), and which modification does not affect, in particular impair, the activity of this sequence. In the case of a binding site having specificity to a selected target antigen, the functionally active variant of an antibody would still have the predetermined binding specificity, though this could be changed e.g., to change the fine specificity to a specific epitope, the affinity, the avidity, the Kon or Koff rate, etc. For example, an affinity matured antibody is specifically understood as a functionally active variant antibody. Hence, the modified CDR sequence in an affinity matured antibody is understood as a functionally active CDR variant.

Specifically, the functionally active variants of an antibody of the invention have the potency to bind O3b-antigen, or the specificity or selectivity to preferentially bind to the O3b-antigen relative to other O3-antigens or any other antigens of K. pneumoniae e.g., binding to O3b-antigen and not binding (or substantially not binding) to the O3a-antigen or O3-antigen of K. pneumoniae, or not significantly binding the O3a-antigen or O3-antigen, and/or not binding to other antigens of K. pneumoniae.

Functionally active variants may be obtained e.g., by changing the sequence of a parent antibody e.g., an antibody comprising the same binding site as the 2F8-G6 antibody, but with modifications within an antibody region besides the binding site, or derived from such parent antibody by a modification within the binding site but that does not impair the antigen binding, and preferably would have substantially the same biological activity as the parent antibody or even an improved activity, including the ability to specifically or selectively bind the O3b-antigen.

Optionally, the functionally active variants may further include a neutralizing potency and/or a potency of complement mediated killing in an SBA assay, and/or optionally further include a potency of an antibody mediated phagocytosis in an OPK assay, and/or optionally further include endotoxin neutralization function in a LAL assay e.g., with substantially the same biological activity, as determined by the specific binding assay or functional test to target K. pneumoniae.

The term “substantially the same” with regard to binding a target antigen or biological activity as used herein refers to the activity as indicated by substantially the same activity being at least 20%, at least 50%, at least 75%, at least 90% e.g., at least 100%, or at least 125%, or at least 150%, or at least 175%, or e.g., up to 200%, or even a higher activity as determined for the comparable or parent antibody.

The preferred variants or derivatives as described herein are functionally active with regard to the antigen binding, preferably which have a potency to specifically bind O3b-antigen, or the specificity or selectivity to preferentially bind to the O3b-antigen relative to other O3-antigens or any other antigens of K. pneumoniae e.g., binding to the O3b-antigen and not binding (or substantially not binding) to the O3a-antigen or O3-antigen of K. pneumoniae, or not significantly binding the O3a-antigen or O3-antigen, and/or not binding to other antigens of K. pneumoniae. Preferred variants are not binding to other antigens of K. pneumoniae, with a Kd value difference of at least 2 logs, preferably at least 3 logs, and optionally further including a potency of complement mediated killing in an SBA assay e.g., to achieve significant reduction in bacterial counts relative to control samples not containing the antibody, and/or optionally further including a potency of an antibody mediated phagocytosis in an OPK assay, such as to achieve significant reduction in bacterial counts relative to control samples not containing the antibody, and/or optionally further including endotoxin neutralization function in a LAL or TLR4 signaling assay, such as to achieve significant reduction of endotoxin activity relative to control samples not containing the antibody e.g., with substantially the same biological activity, as determined by the specific binding assay or functional test to target K. pneumoniae. The significant reduction of activity in the various assays typically means the reduction of at least 50%, preferably at least 60%, 70%, 80%, 90%, 95% or 98% up to complete reduction of about 100% (+/−1%).

In a preferred embodiment the functionally active variant of a parent antibody

a) is a biologically active fragment of the antibody, the fragment comprising at least 50% of the sequence of the molecule, preferably at least 60%, at least 70%, at least 80%, at least 90%, or at least 95% and most preferably at least 97%, 98% or 99%;

b) is derived from the antibody by at least one amino acid substitution, addition and/or deletion, wherein the functionally active variant has a sequence identity to the molecule or part of it, such as an antibody of at least 50% sequence identity, preferably at least 60%, more preferably at least 70%, more preferably at least 80%, still more preferably at least 90%, even more preferably at least 95% and most preferably at least 97%, 98% or 99%; and/or

c) consists of the antibody or a functionally active variant thereof and additionally at least one amino acid or nucleotide heterologous to the polypeptide or the nucleotide sequence.

In one preferred embodiment of the invention, the functionally active variant of the antibody according to the invention is essentially identical to the variant described above, but differs from its polypeptide or the nucleotide sequence, respectively, in that it is derived from a homologous sequence of a different species. These are referred to as naturally occurring variants or analogs.

The term “functionally active variant” also includes naturally occurring allelic variants, as well as mutants or any other non-naturally occurring variants. As is known in the art, an allelic variant is an alternate form of a (poly) peptide that is characterized as having a substitution, deletion, or addition of one or more amino acids that does essentially not alter the biological function of the polypeptide.

Functionally active variants may be obtained by sequence alterations in the polypeptide or the nucleotide sequence e.g., by one or more point mutations, wherein the sequence alterations retains or improves a function of the unaltered polypeptide or the nucleotide sequence, when used in combination of the invention. Such sequence alterations can include, but are not limited to, (conservative) substitutions, additions, deletions, mutations and insertions.

Specific functionally active variants are CDR variants. A CDR variant includes an amino acid sequence modified by at least one amino acid in the CDR region, wherein said modification can be a chemical or a partial alteration of the amino acid sequence, which modification permits the variant to retain the biological characteristics of the unmodified sequence. A partial alteration of the CDR amino acid sequence may be by deletion or substitution of one to several amino acids e.g., 1, 2, 3, 4 or 5 amino acids, or by addition or insertion of one to several amino acids e.g., 1, 2, 3, 4 or 5 amino acids, or by a chemical derivatization of one to several amino acids e.g., 1, 2, 3, 4 or 5 amino acids, or combination thereof. The substitutions in amino acid residues may be conservative substitutions, for example, substituting one hydrophobic amino acid for an alternative hydrophobic amino acid.

Conservative substitutions are those that take place within a family of amino acids that are related in their side chains and chemical properties. Examples of such families are amino acids with basic side chains, with acidic side chains, with non-polar aliphatic side chains, with non-polar aromatic side chains, with uncharged polar side chains, with small side chains, with large side chains etc.

A point mutation is particularly understood as the engineering of a polynucleotide that results in the expression of an amino acid sequence that differs from the non-engineered amino acid sequence in the substitution or exchange, deletion or insertion of one or more single (non-consecutive) or doublets of amino acids for different amino acids.

Preferred point mutations refer to the exchange of amino acids of the same polarity and/or charge. In this regard, amino acids refer to twenty naturally occurring amino acids encoded by sixty-four triplet codons. These 20 amino acids can be split into those that have neutral charges, positive charges, and negative charges:

The “neutral” amino acids are shown below along with their respective three-letter and single-letter code and polarity:

Alanine: (Ala, A) nonpolar, neutral;

Asparagine: (Asn, N) polar, neutral;

Cysteine: (Cys, C) nonpolar, neutral;

Glutamine: (GIn, Q) polar, neutral;

Glycine: (Gly, G) nonpolar, neutral;

Isoleucine: (lile, I) nonpolar, neutral;

Leucine: (Leu, L) nonpolar, neutral;

Methionine: (Met, M) nonpolar, neutral;

Phenylalanine: (Phe, F) nonpolar, neutral;

Proline: (Pro, P) nonpolar, neutral;

Serine: (Ser, S) polar, neutral;

Threonine: (Thr, T) polar, neutral;

Tryptophan: (Trp, W) nonpolar, neutral;

Tyrosine: (Tyr, Y) polar, neutral;

Valine: (Val, V) nonpolar, neutral; and

Histidine: (His, H) polar, positive (10%) neutral (90%).

The “positively” charged amino acids are:

Arginine: (Arg, R) polar, positive; and

Lysine: (Lys, K) polar, positive.

The “negatively” charged amino acids are:

Aspartic acid: (Asp, D) polar, negative; and

Glutamic acid: (Glu, E) polar, negative.

“Percent (%) amino acid sequence identity” with respect to the antibody sequences and homologs described herein is defined as the percentage of amino acid residues in a candidate sequence that are identical with the amino acid residues in the specific polypeptide sequence, after aligning the sequence and introducing gaps, if necessary, to achieve the maximum percent sequence identity, and not considering any conservative substitutions as part of the sequence identity. Those skilled in the art can determine appropriate parameters for measuring alignment, including any algorithms needed to achieve maximal alignment over the full length of the sequences being compared.

An antibody variant is specifically understood to include homologs, analogs, fragments, modifications or variants with a specific glycosylation pattern e.g., produced by glycoengineering, which are functional and may serve as functional equivalents e.g., binding to the specific targets and with functional properties.

An antibody of the present invention may or may not exhibit Fc effector function. Though the mode of action is mainly mediated by neutralizing antibodies without Fc effector functions, Fc can recruit complement and aid elimination of the target antigen, such as a toxin, from the circulation via formation of immune complexes.

Specific antibodies may be devoid of an active Fc moiety, thus, either composed of antibody domains that do not contain an Fc part of an antibody or that do not contain an Fc gamma receptor binding site, or comprising antibody domains lacking Fc effector function e.g., by modifications to reduce Fc effector functions, in particular to abrogate or reduce ADCC and/or CDC activity. Alternative antibodies may be engineered to incorporate modifications to increase Fc effector functions, in particular to enhance ADCC and/or CDC activity.

Such modifications may be effected by mutagenesis e.g., mutations in the Fc gamma receptor binding site or by derivatives or agents to interfere with ADCC and/or CDC activity of an antibody format, so to achieve reduction or increase of Fc effector function.

A significant reduction of Fc effector function is typically understood to refer to Fc effector function of less than 10% of the unmodified (wild-type) format, preferably less than 5%, as measured by ADCC and/or CDC activity. A significant increase of Fc effector function is typically understood to refer to an increase in Fc effector function of at least 10% of the unmodified (wild-type) format, preferably at least 20%, 30%, 40% or 50%, as measured by ADCC and/or CDC activity.

The term “glycoengineered” variants with respect to antibody sequences shall refer to glycosylation variants having modified immunomodulatory (e.g., anti-inflammatory) properties, as a result of the glycoengineering. All antibodies contain carbohydrate structures at conserved positions in the heavy chain constant regions, with each isotype possessing a distinct array of N-linked carbohydrate structures, which variably affect protein assembly, secretion or functional activity. IgG1 type antibodies are glycoproteins that have a conserved N-linked glycosylation site at N297 in each CH2 domain. The two complex bi-antennary oligosaccharides attached to N297 are buried between the CH2 domains, forming extensive contacts with the polypeptide backbone, and their presence is essential for the antibody to bind Fc receptors and mediate effector functions. Removal of N-Glycan at N297 e.g., through mutating N297 e.g., to A, or T299 typically results in aglycosylated antibody formats with reduced ADCC. Specifically, the antibody of the invention may be glycosylated or glycoengineered, or aglycosylated antibodies.

Major differences in antibody glycosylation occur between cell lines, or even with the same cell line grown under different culture conditions. Expression in bacterial cells typically provides for an aglycosylated antibody. CHO cells transfected with genes encoding for the human beta-1,4-galactosyltransferase 1 and beta-galactoside alpha-2,6-sialyltransferase 1 enzymes provide antibodies with galactosylation and alpha-2,6-sialylation. In addition to the choice of host cells, factors that affect glycosylation during recombinant production of antibodies include growth mode, media formulation, culture density, oxygenation, pH, purification schemes and the like.

The term “antigen-binding site” or “binding site” refers to the part of an antibody that participates in antigen binding. The antigen binding site is formed by amino acid residues of the N-terminal variable (“V”) regions of the heavy (“H”) and/or light (“L”) chains, or the variable domains thereof. Three highly divergent stretches within the V regions of the heavy and light chains, referred to as “hypervariable regions”, are inter-posed between more conserved flanking stretches known as framework regions, The antigen-binding site provides for a surface that is complementary to the three-dimensional surface of a bound epitope or antigen, and the hypervariable regions are referred to as “complementarity-determining regions”, or “CDRs.” The binding site incorporated in the CDRs is herein also called “CDR binding site”.

The term “antigen” as used herein interchangeably with the terms “target” or “target antigen” shall refer to a whole target molecule or a fragment of such molecule recognized by an antibody binding site. Specifically, substructures of an antigen e.g., a polypeptide or carbohydrate structure, generally referred to as “epitopes” e.g., B-cell epitopes or T-cell epitope, which are immunologically relevant, may be recognized by such binding site. Specific antigens like the various O3-antigens comprise carbohydrate (mannan) structures and may be provided as isolated antigens optionally provided on an artificial carrier, or else in the form of K. pneumoniae cells expressing the antigens or cell fractions thereof.

The term “epitope” as used herein shall in particular refer to a molecular structure which may completely make up a specific binding partner or be part of a specific binding partner to a binding site of an antibody. An epitope may either be composed of a carbohydrate, a peptidic structure, a fatty acid, an organic, biochemical or inorganic substance or derivatives thereof and any combinations thereof. If an epitope is comprised in a peptidic structure, such as a peptide, a polypeptide or a protein, it will usually include at least 3 amino acids, preferably 5 to 40 amino acids, and more preferably between about 10-20 amino acids. Epitopes can be either linear or conformational epitopes. A linear epitope is comprised of a single segment of a primary sequence of a polypeptide or carbohydrate chain. Linear epitopes can be contiguous or overlapping.

Conformational epitopes are comprised of amino acids or carbohydrates brought together by folding the polypeptide to form a tertiary structure and the amino acids are not necessarily adjacent to one another in the linear sequence. Specifically and with regard to polypeptide antigens a conformational or discontinuous epitope is characterized by the presence of two or more discrete amino acid residues, separated in the primary sequence, but assembling to a consistent structure on the surface of the molecule when the polypeptide folds into the native protein/antigen.

Herein the term “epitope” shall particularly refer to the single epitope recognized by an antibody, or a cross-reactive epitope which is shared by at least two different antigens and optionally recognized by the cross-reactive antibody.

The term “expression” is understood in the following way. Nucleic acid molecules containing a desired coding sequence of an expression product such as e.g., an antibody as described herein, and control sequences such as e.g., a promoter in operable linkage, may be used for expression purposes. Hosts transformed or transfected with these sequences are capable of producing the encoded proteins. In order to effect transformation, the expression system may be included in a vector; however, the relevant DNA may also be integrated into the host chromosome. Specifically the term refers to a host cell and compatible vector under suitable conditions e.g., for the expression of a protein coded for by foreign DNA carried by the vector and introduced to the host cell.

Coding DNA is a DNA sequence that encodes a particular amino acid sequence for a particular polypeptide or protein such as e.g., an antibody. Promoter DNA is a DNA sequence which initiates, regulates, or otherwise mediates or controls the expression of the coding DNA. Promoter DNA and coding DNA may be from the same gene or from different genes, and may be from the same or different organisms. Recombinant cloning vectors will often include one or more replication systems for cloning or expression, one or more markers for selection in the host e.g., antibiotic resistance, and one or more expression cassettes.

“Vectors” used herein are defined as DNA sequences that are required for the transcription of cloned recombinant nucleotide sequences, i.e. of recombinant genes and the translation of their mRNA in a suitable host organism.

An “expression cassette” refers to a DNA coding sequence or segment of DNA that code for an expression product that can be inserted into a vector at defined restriction sites. The cassette restriction sites are designed to ensure insertion of the cassette in the proper reading frame. Generally, foreign DNA is inserted at one or more restriction sites of the vector DNA, and then is carried by the vector into a host cell along with the transmissible vector DNA. A segment or sequence of DNA having inserted or added DNA, such as an expression vector, can also be called a “DNA construct”.

Expression vectors comprise the expression cassette and additionally usually comprise an origin for autonomous replication in the host cells or a genome integration site, one or more selectable markers (e.g., an amino acid synthesis gene or a gene conferring resistance to antibiotics such as zeocin, kanamycin, G418 or hygromycin), a number of restriction enzyme cleavage sites, a suitable promoter sequence and a transcription terminator, which components are operably linked together. The term “vector” as used herein includes autonomously replicating nucleotide sequences as well as genome integrating nucleotide sequences. A common type of vector is a “plasmid”, which generally is a self-contained molecule of double-stranded DNA that can readily accept additional (foreign) DNA and which can readily be introduced into a suitable host cell. A plasmid vector often contains coding DNA and promoter DNA and has one or more restriction sites suitable for inserting foreign DNA. Specifically, the term “vector” or “plasmid” refers to a vehicle by which a DNA or RNA sequence (e.g., a foreign gene) can be introduced into a host cell, so as to transform the host and promote expression (e.g., transcription and translation) of the introduced sequence.

The term “host cell” as used herein shall refer to primary subject cells transformed to produce a particular recombinant protein, such as an antibody as described herein, and any progeny thereof. It should be understood that not all progeny are exactly identical to the parental cell (due to deliberate or inadvertent mutations or differences in environment), however, such altered progeny are included in these terms, so long as the progeny retain the same functionality as that of the originally transformed cell. The term “host cell line” refers to a cell line of host cells as used for expressing a recombinant gene to produce recombinant polypeptides such as recombinant antibodies. The term “cell line” as used herein refers to an established clone of a particular cell type that has acquired the ability to proliferate over a prolonged period of time. Such host cell or host cell line may be maintained in cell culture and/or cultivated to produce a recombinant polypeptide.

The term “isolated” or “isolation” as used herein with respect to a nucleic acid, an antibody or other compound shall refer to such compound that has been sufficiently separated from the environment with which it would naturally be associated, so as to exist in “substantially pure” form. “Isolated” does not necessarily mean the exclusion of artificial or synthetic mixtures with other compounds or materials, or the presence of impurities that do not interfere with the fundamental activity, and that may be present, for example, due to incomplete purification. In particular, isolated nucleic acid molecules of the present invention are also meant to include those which are not naturally occurring e.g., codon-optimized nucleic acids or cDNA, or chemically synthesized.

Likewise, the isolated antibody of the invention is specifically non-naturally occurring e.g., as provided in a combination preparation with another antibody or active agent, which combination does not occur in nature, or an optimized or affinity-maturated variant of a naturally occurring antibody, or an antibody with a framework-region which is engineered to improve the manufacturability of the antibody. By such optimizing or engineering the antibody comprises one or more synthetic sequences or characteristics, which would not be found in the context of the antibody in nature.

With reference to nucleic acids of the invention, the term “isolated nucleic acid” is sometimes used. This term, when applied to DNA, refers to a DNA molecule that is separated from sequences with which it is immediately contiguous in the naturally occurring genome of the organism in which it originated. For example, an “isolated nucleic acid” may comprise a DNA molecule inserted into a vector, such as a plasmid or virus vector, or integrated into the genomic DNA of a prokaryotic or eukaryotic cell or host organism. When applied to RNA, the term “isolated nucleic acid” refers primarily to an RNA molecule encoded by an isolated DNA molecule as defined above. Alternatively, the term may refer to an RNA molecule that has been sufficiently separated from other nucleic acids with which it would be associated in its natural state (i.e., in cells or tissues). An “isolated nucleic acid” (either DNA or RNA) may further represent a molecule produced directly by biological or synthetic means and separated from other components present during its production.

With reference to polypeptides or proteins, such as isolated antibodies or epitopes of the invention, the term “isolated” shall specifically refer to compounds that are free or substantially free of material with which they are naturally associated such as other compounds with which they are found in their natural environment, or the environment in which they are prepared (e g. cell culture) when such preparation is by recombinant DNA technology practiced in vitro or in vivo. Isolated compounds can be formulated with diluents or adjuvants and still for practical purposes be isolated—for example, the polypeptides or polynucleotides can be mixed with pharmaceutically acceptable carriers or excipients when used in diagnosis or therapy. In particular, the isolated antibody of the invention differs from polyclonal serum preparations raised against K. pneumoniae strains, because it is provided in the isolated and purified form, preferably provided in a preparation comprising the isolated antibody as the only active substance. This does not preclude, however, that the isolated antibody is provided in a combination product comprising a limited number of further well-defined (isolated) antibodies. Isolated antibodies may as well be provided on a solid, semi-liquid or liquid carrier, such as beads.

The term “neutralizing” or “neutralization” is used herein in the broadest sense and refers to any molecule that inhibits a pathogen, such as K. pneumoniae from infecting a subject, or to inhibit the pathogen from promoting infections by producing endotoxins, or to inhibit the endotoxins from exerting their biological activity, irrespective of the mechanism by which neutralization is achieved. Neutralization can be achieved, e.g., by an antibody that inhibits the colonization by K. pneumoniae of mucosal surfaces, invasion to sterile body sites, and eliciting adverse biological signals (in worst case inducing septic shock) in the host.

In the strict sense neutralization means, inhibiting the binding of specific LPS to its cognate receptor (e.g., Toll-like receptor-4 complex) and hence eliciting biological activity. This neutralization potency is typically determined in a standard assay e.g., an in vitro or in vivo neutralization assay e.g., a LAL test, or TLR-4 based assays, where the inhibition of endotoxin's biological activity is measured e.g., by colorimetry.

Antibodies combating or neutralizing K. pneumoniae are interfering with the pathogens and pathogenic reactions, thus able to limit or prevent infection and/or to ameliorate a disease condition resulting from such infection, or to inhibit K. pneumoniae pathogenesis, in particular dissemination and replication into or within sterile body compartments/sites of the host. In this regard the neutralizing antibody is also understood as being a “protective antibody” meaning that the antibody is responsible for immunity to an infectious agent observed in active or passive immunity. In particular, neutralizing or protective antibodies as described herein are possibly used for therapeutic purposes e.g., for prophylaxis or therapy, to prevent, ameliorate, treat or at least partially arrest disease symptoms, side effects or progression of disease induced by a pathogen. Specifically, protective antibodies are able to kill or impede replication of live K. pneumoniae cells by e.g., inducing serum bactericidal or opsonophagocytic activities, or remove whole bacterial cells or the LPS molecules thereof from the sterile body sites following therapeutic applications (i.e. given on an established infection). Alternatively, prophylactically applied protective antibodies inhibit establishment of an infection (i.e. spread of K. pneumoniae from non-sterile sites to sterile body compartments) by one of the abovementioned or other mechanisms.

The term “biological sample” as used herein shall refer to any material obtained from a subject, such as a human being, that contains, or potentially contains, biological material which could contain K. pneumoniae. The biological sample can be a tissue, fluid or cell culture sample. Examples of samples for use in accordance with the invention include, but are not limited to patient samples e.g., tissue or body fluids, specifically a respiratory tract specimen such as endotracheal aspirates, pleural fluid, lung tap, nasal swab or sputum, a blood sample, stool sample, skin and urine sample or cerebrospinal fluid.

The biological sample typically comprises a complex biological matrix such as complex viscous biological fluids containing multiple types of biological and small organic molecules, for example mucous exudates rich in protein matter. Suitable additives or extraction procedures may be used to reduce the non-specific binding that can be associated with a matrix in the sample and/or lower the matrix viscosity by solubilizing and/or breaking down viscous or solid components of the sample matrix. Sample preparation methods may be employed that liberate markers from organisms and/or break down and/or liquefy biological matrices. Biological matrices that may be analyzed include mucus-containing samples such as nasal secretions, sputum, phlegm, pharyngeal exudates, urethral or vaginal secretions, and washes of such membrane surfaces.

Suitable sample preparation methods include method steps to reduce the effect of the biological matrix on the assay. Such method steps may include but are not limited to, e.g., capture, chromatography, spin-centrifugation and dialysis.

The material obtained from a subject may also be in the form of bacterial isolates e.g., in the form of a cell culture for cultivating the isolated K. pneumoniae or a cell culture product. Culture media may be selective to enrich solely the K. pneumoniae population, or non-selective.

Bacterial isolate preparation typically involves an incubating step to maintain the sample in conditions that enhance the proliferation of K. pneumoniae, thereby enriching the K. pneumoniae population in the sample.

Once the isolate is obtained, the bacterium may be further investigated by biochemical and/or serological tests e.g., to determine the O type, and the level of O3b-antigen expressed. Several typing methods are available to study K. pneumoniae strains. These methods typically include serotyping, toxin-typing, standard typing for genetic relationship/phylogeny including multi-locus sequence typing (MLST), or Pulsed Field Gel Electrophoresis (PFGE).

The term “O3b-antigen” also referred to as “O3b-type” as used herein shall refer to the (methyl phosphate containing) carbohydrate structure of the LPS O-antigen of K. pneumoniae depicted in Formula (I), in particular comprising a mannan polymer and a structure comprising at least one of the trimannose repeating unit included in Formula (I). Such structure and the trimannose repeating unit has heretofore not been identified. The structure is similar, but distinct from that of the O3a-antigen (Formula (II)) or O3-antigen (Formula (III)). It is thus, surprising, that the newly identified O3b-structure comprises a distinct epitope. Antibodies specifically recognizing the pentamannose structure of the O3-antigen were previously found not to recognize the O3a-antigen (tetramannose structure). The minimum number of mannose residues needed to define the O3a and O3 polysaccharide was described in the prior art to be four, and the shortest candidate for an antibody epitope was found to be the tetramannan (see (7)). O3b is herein understood as a new serotype determinant, which is similar, but distinct from the O3a or O3 serotype that is characterized by the presence of the other O3-antigens and the absence of the O3b (trimannose) structure.

The respective O-antigen comprising the O3b structure is herein referred to as “O3b-antigen” which includes the “O3b-epitope” being recognized by a O3b-specific antibody of the invention. The O3b-antigen is understood as the outer part of the LPS of K. pneumoniae of the O3b O-type, which is the surface accessible antigenic carbohydrate structure comprising one or more specific O3b-epitopes incorporated therein.

The genotype of K. pneumoniae of the O3b-type is specifically characterized by low homology in sequence of genes wbdD and wbdA compared to corresponding genes in the rfb operon of O3 and O3a-type K. pneumoniae strains.

Any K. pneumoniae which is characterized by a LPS O-antigen comprising at least one O3b structure is herein referred to as K. pneumoniae of the O3b-type. LPS of K. pneumoniae of the O3b-type may comprise the O3b structure, or both, O3b and O3a and/or O3 structures.

The O3a-antigen is understood as the outer part of the LPS of K. pneumoniae of the O3a-type, which is the surface accessible antigenic carbohydrate structure comprising one or more specific O3a-epitopes incorporated therein, and which does not include any O3b-structure.

The O3-antigen is understood as the outer part of the LPS of K. pneumoniae of the O3-type, which is the surface accessible antigenic carbohydrate structure comprising one or more specific O3-epitopes incorporated therein, and which does not include any O3b-structure or O3a-structure.

The term “O3-antigens” as used herein shall refer to all of the O3b, the O3a, and the O3-antigens. When comparing the O3b-antigen to the other O3-antigen(s), the other O3-antigen(s) shall refer to the O3a-antigen and/or the O3 antigen.

The term “pan-O3” with respect to target antigens recognized by a “pan-O3-antibody” as used herein shall refer to all of the O3b-antigen, the O3a-antigen and the O3 antigen, and the cross-reactive, yet O3-specific, antibody recognizing each of the O3b-antigen, the O3a-antigen and the O3 antigen.

“Specific” binding, recognizing or targeting as used herein, means that the binder e.g., antibody or antigen-binding portion thereof, exhibits appreciable affinity for the target antigen or a respective epitope in a heterogeneous population of molecules. Thus, under designated conditions (e.g., immunoassay), a binder specifically binds to the target O3b antigen and does not bind in a significant amount to other molecules present in a sample. The specific binding means that binding is selective in terms of target identity, high, medium or low binding affinity or avidity, as selected. Selective binding is usually achieved if the binding constant or binding dynamics is at least 10-fold different (understood as at least 1 log difference), preferably the difference is at least 100-fold (understood as at least 2 logs difference), and more preferred a least 1000-fold (understood as at least 3 logs difference) as compared to another target.

The term “specificity” is also understood to apply to binders which bind to one or more molecules e.g., cross-specific binders. Preferred cross-specific (also called polyspecific or cross-reactive) binders targeting at least two different targets or epitopes or nucleotide sequences of such targets or targeting a cross-reactive epitope or nucleotide sequence on at least two different targets, specifically bind the targets with substantially similar binding affinity e.g., with less than 100-fold difference or even less than 10-fold difference, or, with substantially different binding affinity e.g., with at least 10 fold or at least 100 fold difference. The cross-specific binder which recognizes both, a first (e.g., O3b-antigen) and a second (e.g., the O3a-antigen and optionally also the O3 antigen) target, which preferentially binds the first target over the second target is typically characterized by equal affinities or a higher affinity to the first target relative to the second one, specifically wherein the differential binding affinity to preferentially bind the first antigen relative to the second antigen is specifically at least equal or more than equal e.g., at least 1.5 fold, or at least 2-fold, or at least 3-fold, or at least 4-fold, or at least 5 fold, or at least 6-fold, or at least 7-fold, or at least 8-fold, or at least 9-fold, or at least 10-fold higher. Such differential binding may be determined by an immunoassay, preferably immunoblotting, ELISA or other immunological methods.

Preferred antibodies of the invention are binding the O3b-antigen (only O3b, or preferentially binding O3b relative to the O3a-antigen and/or the O3-antigen), with a high affinity, in particular with a high on and/or a low off rate, or a high avidity of binding. The binding affinity of an antibody is usually characterized in terms of the concentration of the antibody, at which half of the antigen binding sites are occupied, known as the dissociation constant (Kd, or K_{D}). Usually a binder is considered a high affinity binder with a Kd<10⁻⁷ M, in some cases e.g., for therapeutic purposes with higher affinities e.g., with a Kd<10⁻⁸ M, preferably a Kd<10⁻⁹ M, even more preferred is a Kd<10-10 M.

Yet, in a particularly preferred embodiment the individual antigen binding affinities are of medium affinity e.g., with a Kd of less than 10⁻⁶ and up to 10⁻⁸ M e.g., when binding to at least two antigens.

Medium affinity binders may be provided according to the invention, preferably in conjunction with an affinity maturation process, if necessary.

Affinity maturation is the process by which antibodies with increased affinity for a target antigen are produced. Any one or more methods of preparing and/or using affinity maturation libraries available in the art may be employed in order to generate affinity matured antibodies in accordance with various embodiments of the invention disclosed herein. Exemplary such affinity maturation methods and uses, such as random mutagenesis, bacterial mutator strains passaging, site-directed mutagenesis, mutational hotspots targeting, parsimonious mutagenesis, antibody shuffling, light chain shuffling, heavy chain shuffling, CDR1 and/or CDR1 mutagenesis, and methods of producing and using affinity maturation libraries amenable to implementing methods and uses in accordance with various embodiments of the invention disclosed herein, include, for example, those disclosed in: Prassler et al. (2009); Immunotherapy, Vol. 1(4), pp. 571-583; Sheedy et al. (2007), Biotechnol. Adv., Vol. 25(4), pp. 333-352; WO2012/009568; WO2009/036379; WO2010/05256; US2002/0177170; WO2003/O74679.

With structural changes of an antibody, including amino acid mutagenesis or as a consequence of somatic mutation in immunoglobulin gene segments, variants of a binding site to an antigen are produced and selected for greater affinities. Affinity matured antibodies may exhibit a several logfold greater affinity than a parent antibody. Single parent antibodies may be subject to affinity maturation. Alternatively pools of antibodies with similar binding affinity to the target antigen may be considered as parent structures that are varied to obtain affinity matured single antibodies or affinity matured pools of such antibodies.

The preferred affinity maturated variant of an antibody according to the invention exhibits at least a 2 fold increase in affinity of binding, preferably at least a 5, preferably at least 10, preferably at least 50, or preferably at least 100 fold increase. The affinity maturation may be employed in the course of the selection campaigns employing respective libraries of parent molecules, either with antibodies having medium binding affinity to obtain the antibody of the invention having the specific target binding property of a binding affinity Kd<10⁻⁸ M. Alternatively, the affinity may be even more increased by affinity maturation of the antibody according to the invention to obtain the high values corresponding to a Kd of less than 10⁻⁹ M, preferably less than 10⁻¹⁰ M or even less than 10⁻¹¹ M, most preferred in the picomolar range.

In certain embodiments binding affinity is determined by an affinity ELISA assay. In certain embodiments binding affinity is determined by a BIAcore, ForteBio or MSD assays. In certain embodiments binding affinity is determined by a kinetic method. In certain embodiments binding affinity is determined by an equilibrium/solution method.

Use of the term “having the same specificity”, “having the same binding site” or “binding the same epitope” indicates that equivalent monoclonal antibodies exhibit the same or essentially the same, i.e. similar immunoreaction (binding) characteristics and compete for binding to a pre-selected target binding sequence. The relative specificity of an antibody molecule for a particular target can be relatively determined by competition assays e.g., as described in Harlow, et al., ANTIBODIES: A LABORATORY MANUAL, Cold Spring Harbor Laboratory Press, Cold Spring Harbor, N.Y., 1988).

The term “compete”, as used herein with regard to an antibody, means that a first antibody, or an antigen-binding portion thereof, binds to an epitope in a manner sufficiently similar to the binding of a second antibody, or an antigen-binding portion thereof, such that the result of binding of the first antibody with its cognate epitope is detectably decreased in the presence of the second antibody compared to the binding of the first antibody in the absence of the second antibody. The alternative, where the binding of the second antibody to its epitope is also detectably decreased in the presence of the first antibody, can, but need not be the case. That is, a first antibody can inhibit the binding of a second antibody to its epitope without that second antibody inhibiting the binding of the first antibody to its respective epitope. However, where each antibody detectably inhibits the binding of the other antibody with its cognate epitope, whether to the same, greater, or lesser extent, the antibodies are said to “compete” with each other for binding of their respective epitope(s). Antibodies that compete with any of the exemplified antibodies for binding the O3b-antigen are particularly encompassed by the present invention.

“Competitively binding” or Competition herein means a greater relative inhibition than about 30% as determined by competition ELISA analysis or by ForteBio analysis. It may be desirable to set a higher threshold of relative inhibition as criteria of what is a suitable level of competition in a particular context e.g., where the competition analysis is used to select or screen for new antibodies designed with the intended function of the binding of the antigen. Thus, for example, it is possible to set criteria for the competitive binding, wherein at least 40% relative inhibition is detected, or at least 50%, at least 60%, at least 70%, at least 80%, at least 90% or even at least 100%, before an antibody is considered sufficiently competitive.

The term “diagnostic kit” as used herein refers to a kit or set of parts, which in combination or mixture can be used to carry out the measurement/detection of one or more analytes or markers to determine a disease or disease condition, or to predict the disease or the disease progression. In particular, the kit contains at least a detection molecule and/or a binder, wherein the detection molecule and/or the binder specifically recognizes the analyte or marker, or a reaction product of such analyte or marker. In addition, various reagents or tools may be included in the kit. The diagnostic kit may comprise any useful reagents for carrying out the subject methods, including substrates such as microbeads or planar arrays or wells, reagents for biomarker isolation, detection molecules directed to specific targets, reagents such as primers for nucleic acid sequencing or amplification, arrays for nucleic acid hybridization, detectable labels, solvents or buffers and the like, various linkers, various assay components, blockers, and the like.

A kit may also include instructions for use in a diagnostic method. Such instructions can be, for example, provided on a device included in the kit e.g., tools or a device to prepare a biological sample for diagnostic purposes, such as separating a cell and/or protein containing fraction before determining a marker. The kit may conveniently be provided in the storage stable form, such as a commercial kit with a shelf-life of at least 6 months.

Specific diagnostic kits also comprise a solid support comprising a detection molecule or having an immobilized patterned array of detection molecules directed against markers of interest, preferably including a first region containing a first binding reagent directed against a first marker and a second region containing a second binding reagent directed against a second marker.

In particular, a sandwich format can be used. For example, one or more binder is conjugated to a substrate prior to the contacting with a biological sample. The one or more binder may be conjugated to a detectable label to serve as a detection molecule. In other embodiments, the one or more binder is conjugated to a detectable label. In this configuration, the one or more binders may be conjugated to a substrate prior to the contacting with the biological sample to serve as a capture agent. Furthermore, the one or more binder can be conjugated to a substrate prior to the contacting with the biological sample, and/or the one or more binder is conjugated to a detectable label. In such cases, the one or more binder can act as either or both of a capture agent and a detection agent.

The diagnostic kit is specifically provided for use in an immunoassay, wherein the detection molecule is a specific binder that binds to the analyte or marker by an immunoreaction. Such binder may be antibodies or antibody fragments or antibody-like scaffolds binding to a target antigen.

Suitable immunoassays are any of ELISA, CIA, RIA, IRMA, agglutination assay, immunochromatography, dipstick assay and Western-blot.

The term “K. pneumoniae infection” and “K. pneumoniae colonization” is understood in the following way: Klebsiella pneumoniae is a Gram-negative, bacterium that is a member of the family Enterobacteriaceae. It is a ubiquitous bacterium, which can also colonize the human host, typically in the intestines or the upper airways. Being an opportunistic pathogen, from these sites it can invade sterile body sites in case not properly controlled by the immune system. Uncontrolled bacterial replication at these sites will induce inflammation, in a great part, mediated by the endotoxin (i.e. LPS) molecules released from K. pneumoniae. In case of bacteremia, endotoxin molecules may trigger septic shock.

K. pneumoniae colonization means that the subject has a sufficiently high concentration of K. pneumoniae bacteria at a site that they can be detected, yet the bacteria are causing no signs or symptoms. Colonization can persist for a long period of time, with resolution influenced by the immune response to the organism, competition at the site from other organisms and, sometimes, use of antimicrobials.

In general, bacteremia caused by K. pneumoniae may be successfully treated with known conventional antibacterial therapy, such as treatment with antibiotics, steroid and non-steroid inhibitors of inflammation. The present invention provides for a new immunotherapy, employing antibodies specifically recognizing K. pneumoniae, which is optionally combined with anti-bacterial or anti-inflammatory therapy. Exemplary antibiotics used for treating patients with K. pneumoniae infection are aminoglycosides, cephalosporines, aminopenicilines, carbapenems, fluoroquinolons, tygecycline, colistin, etc.

Multi-drug resistant (MDR) K. pneumoniae is particularly understood as those strains demonstrating resistance to three or more classes of antibiotics e.g., the following agents/groups: penicillins, cephalosporins, carbapenems, aminoglycosides, tetracyclines, fluoroquinolones, nitrofurantoin, trimethoprim (and its combinations), fosfomycin, polymixins, chloramphenicol, azthreonam, or tigecycline.

With the recent emergence of antibiotic-resistant strains, treating bacteremia of this nature has become significantly more difficult. Patients who develop K. pneumoniae disease have longer hospital and ICU stays, high mortality, and greater health care costs than patients without K. pneumoniae disease. Patient care may be improved and nosocomial infections may be reduced by preventing, rather than treating, K. pneumoniae disease prophylaxis when a patient is heavily colonized by K. pneumoniae.

K. pneumoniae disease is specifically understood as a disease caused by K. pneumoniae infection. Such diseases include local and systemic disease. Severe cases of disease are e.g., primary and secondary bacteremia, pneumonia, urinary tract infection, liver abscess, peritonitis, or meningitis.

The term “recombinant” as used herein shall mean “being prepared by or the result of genetic engineering”. A recombinant host specifically comprises an expression vector or cloning vector, or it has been genetically engineered to contain a recombinant nucleic acid sequence, in particular employing nucleotide sequence foreign to the host. A recombinant protein is produced by expressing a respective recombinant nucleic acid in a host. The term “recombinant antibody”, as used herein, includes antibodies that are prepared, expressed, created or isolated by recombinant means, such as (a) antibodies isolated from an animal (e.g., a mouse) that is transgenic or transchromosomal for human immunoglobulin genes or a hybridoma prepared therefrom, (b) antibodies isolated from a host cell transformed to express the antibody e.g., from a transfectoma, (c) antibodies isolated from a recombinant, combinatorial human antibody library or library of antigen-binding sequences of an antibody, and (d) antibodies prepared, expressed, created or isolated by any other means that involve splicing of human immunoglobulin gene sequences to other DNA sequences. Such recombinant antibodies comprise antibodies engineered to include rearrangements and mutations which occur, for example, during antibody maturation. In accordance with the present invention there may be employed conventional molecular biology, microbiology, and recombinant DNA techniques within the skill of the art. Such techniques are explained fully in the literature. See, e.g., Maniatis, Fritsch & Sambrook, “Molecular Cloning: A Laboratory Manual, Cold Spring Harbor, (1982).

Selective binding can be further improved by recombinant antibody optimization methods known in the art. For example, certain regions of the variable regions of the immunoglobulin chains described herein may be subjected to one or more optimization strategies, including light chain shuffling, destinational mutagenesis, CDR amalgamation, and directed mutagenesis of selected CDR and/or framework regions.

The term “subject” as used herein shall refer to a warm-blooded mammalian, particularly a human being or a non-human animal. K. pneumoniae is a critically important human pathogen that is also an emerging concern in veterinary medicine. It is present in a wide range of non-human animal species. Thus, the term “subject” may also particularly refer to animals including dogs, cats, rabbits, horses, cattle, pigs and poultry. In particular the medical use of the invention or the respective method of treatment applies to a subject in need of prophylaxis or treatment of a disease condition associated with a K. pneumoniae infection. The subject may be a patient at risk of a K. pneumoniae infection or suffering from disease, including early stage or late stage disease. The term “patient” includes human and other mammalian subjects that receive either prophylactic or therapeutic treatment. The term “treatment” is thus meant to include both prophylactic and therapeutic treatment.

A subject is e.g., treated for prophylaxis or therapy of K. pneumoniae disease conditions. In particular, the subject is treated, which is either at risk of infection or developing such disease or disease recurrence, or a subject that is suffering from such infection and/or disease associated with such infection.

Specifically the term “prophylaxis” refers to preventive measures which is intended to encompass prevention of the onset of pathogenesis or prophylactic measures to reduce the risk of pathogenesis.

Specifically, the treatment may be by interfering with the pathogenesis of K. pneumoniae as causal agent of the condition,

The term “substantially pure” or “purified” as used herein shall refer to a preparation comprising at least 50% (w/w), preferably at least 60%, 70%, 80%, 90% or 95% of a compound, such as a nucleic acid molecule or an antibody. Purity is measured by methods appropriate for the compound (e.g., chromatographic methods, polyacrylamide gel electrophoresis, HPLC analysis, and the like).

The term “therapeutically effective amount”, used herein interchangeably with any of the terms “effective amount” or “sufficient amount” of a compound e.g., an antibody of the present invention, is a quantity or activity sufficient to, when administered to the subject effect beneficial or desired results, including clinical results, and, as such, an effective amount or synonym thereof depends upon the context in which it is being applied.

An effective amount is intended to mean that amount of a compound that is sufficient to treat, prevent or inhibit such diseases or disorder. In the context of disease, therapeutically effective amounts of the antibody as described herein are specifically used to treat, modulate, attenuate, reverse, or affect a disease or condition that benefits from an inhibition of K. pneumoniae pathogenesis, for example, adhesion and colonization of mucosal surfaces, uncontrolled replication within sterile body sites, and toxicity of host cells by bacterial products.

The amount of the compound that will correspond to such an effective amount will vary depending on various factors, such as the given drug or compound, the pharmaceutical formulation, the route of administration, the type of disease or disorder, the identity of the subject or host being treated, and the like, but can nevertheless be routinely determined by one skilled in the art.

A therapeutically effective amount of the antibody as described herein, such as provided to a human patient in need thereof, may specifically be in the range of 0.5-50 mg/kg, preferably 5-40 mg/kg, even more preferred up to 20 mg/kg, up to 10 mg/kg, up to 5 mg/kg, though higher doses may be indicated e.g., for treating acute disease conditions. The dose can be much lower if a highly potent antibody is used. In such case, the effective amount may be in the range of 0.005 to 5 mg/kg, preferably 0.05 to 1 mg/kg.

Moreover, a treatment or prevention regime of a subject with a therapeutically effective amount of the antibody of the present invention may consist of a single administration, or alternatively comprise a series of applications. For example, the antibody may be administered at least once a year, at least once a half-year or at least once a month. However, in another embodiment, the antibody may be administered to the subject from about one time per week to about a daily administration for a given treatment. The length of the treatment period depends on a variety of factors, such as the severity of the disease, either acute or chronic disease, the age of the patient, the concentration and the activity of the antibody format. It will also be appreciated that the effective dosage used for the treatment or prophylaxis may increase or decrease over the course of a particular treatment or prophylaxis regime. Changes in dosage may result and become apparent by standard diagnostic assays known in the art. In some instances, chronic administration may be required.

Monoclonal antibodies (mAbs) highly specific to O3b, and in particular the pan-O3 antibodies have great potential as diagnostic reagents for the identification of Klebsiella pneumoniae. Furthermore, in particular following humanization, these mAbs are suitable to be used for the prophylaxis (e.g., for high risk groups) and treatment of K. pneumoniae infections.

The O3 serogroup used to be considered a single uniform structure, however, it surprisingly turned out to comprise three different subtypes, which differ in the number of mannose residues within the repeating units. These alterations in the structure represent antigenic differences as proven by the reactivity pattern of monoclonal antibodies specific to the O3 serogroup. Selection of monoclonal antibodies cross-reacting with all 3 different serotypes within the O3 serogroup is of high relevance with respect to putative product development.

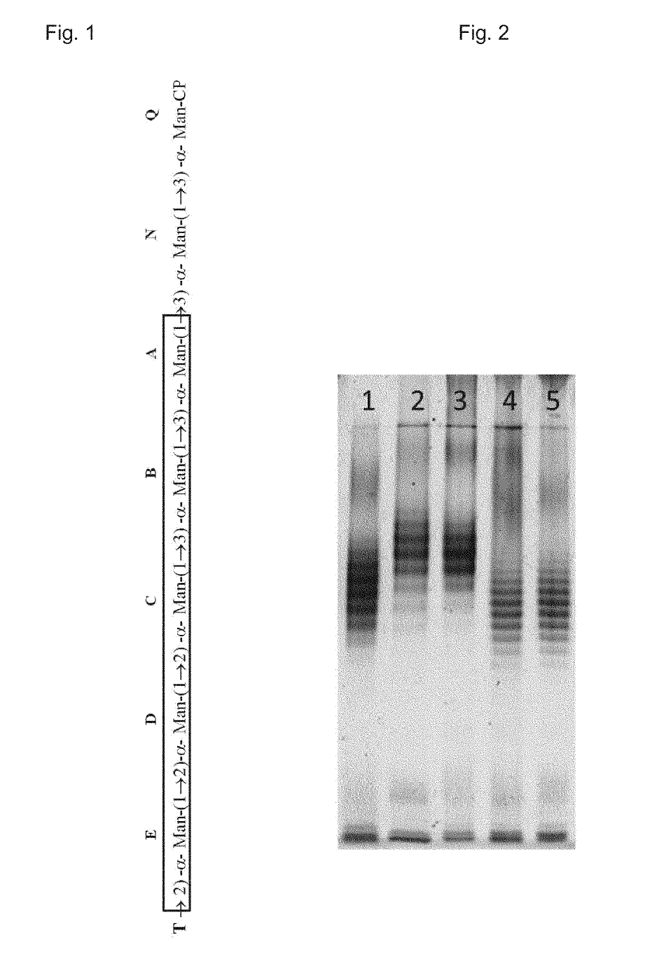

Genetic analysis of the corresponding genes encoding mannose polymerization, i.e. synthesis of the O-antigen subunits revealed differences that justify the observed structural alterations. Based on the genetic and phenotypic differences we propose the newly described serotypes to be termed O3a and O3b in order to differentiate them from the published “classical” penta-mannose structure, known as the O3 serotype (FIG. 1—published in (3)).

Screening of a collection of over 150 clinical isolates showed that the newly described O3b serotype is expressed by more isolates than any other mannan types (i.e., O3, O3a and O5) together.

Structural diversity of O-antigen repeating units is herein suggested among O3 serogroup K. pneumoniae strains based on LPS separation pattern. Genetic heterogeneity within the rfb operon encoding O3 side chains corroborated the proposed structural differences. mAbs were selected that are either specific to one of these variants or cross-react with all three types within the O3 serogroup.

Preliminary data of biochemical structural analysis implied that the penta-mannose repeating unit structure is replaced by tetra- and tri-saccharide repeat units and hence represent distinct antigenic structures; herewith designated as serotypes O3a and O3b.

The Klebsiella O3a subunit is identical to that of E. coli O9a, however, the O3b antigen represents a novel structure, and therefore antibodies specific to O3b as well as those cross-reacting between O3, O3a and O3b are valuable means for e.g., immunotherapy and/or immunodiagnostics. The O3 cross-reactive (pan-O3 reactive) mAb is specific to almost a quarter (23.2%) of all Klebsiella isolates, and hence further development of such mAbs as diagnostic and/or therapeutic product candidate may be justified.

Once antibodies with the desired binding properties are identified, such antibodies, including antibody fragments can be produced by methods well-known in the art, including, for example, hybridoma techniques or recombinant DNA technology.

Recombinant monoclonal antibodies can, for example, be produced by isolating the DNA encoding the required antibody chains and transfecting a recombinant host cell with the coding sequences for expression, using well known recombinant expression vectors, e.g., the plasmids of the invention or expression cassette(s) comprising the nucleotide sequences encoding the antibody sequences. Recombinant host cells can be prokaryotic and eukaryotic cells, such as those described above.

According to a specific aspect, the nucleotide sequence may be used for genetic manipulation to humanize the antibody or to improve the affinity, or other characteristics of the antibody. For example, the constant region may be engineered to more nearly resemble human constant regions to avoid immune response, if the antibody is used in clinical trials and treatments in humans. It may be desirable to genetically manipulate the antibody sequence to obtain greater affinity to the O3b target and greater efficacy against Klebsiella pneumoniae. It will be apparent to one of skill in the art that one or more polynucleotide changes can be made to the antibody and still maintain its binding ability to the target O3b-antigen.

The production of antibody molecules, by various means, is generally well understood. U.S. Pat. No. 6,331,415 (Cabilly et al.), for example, describes a method for the recombinant production of antibodies where the heavy and light chains are expressed simultaneously from a single vector or from two separate vectors in a single cell. Wibbenmeyer et al., (1999, Biochim Biophys Acta 1430(2):191-202) and Lee and Kwak (2003, J. Biotechnology 101:189-198) describe the production of monoclonal antibodies from separately produced heavy and light chains, using plasmids expressed in separate cultures of host cells. Various other techniques relevant to the production of antibodies are provided in, e.g., Harlow, et al., ANTIBODIES: A LABORATORY MANUAL, Cold Spring Harbor Laboratory Press, Cold Spring Harbor, N.Y., (1988).

If desired, the antibody of the invention, e.g., the 2F8-G6 antibody or a functional variant thereof, may be sequenced and the polynucleotide sequence may then be cloned into a vector for expression or propagation. The sequence encoding the antibody may be maintained in vector in a host cell and the host cell can then be expanded and frozen for future use. Production of recombinant monoclonal antibodies in cell culture can be carried out through cloning of antibody genes from B cells by means known in the art.

In another aspect, the invention provides an isolated nucleic acid comprising a sequence that codes for production of the recombinant antibody of the present invention.

An antibody encoding nucleic acid can have any suitable characteristics and comprise any suitable features or combinations thereof. Thus, for example, an antibody encoding nucleic acid may be in the form of DNA, RNA, or a hybrid thereof, and may include non-naturally-occurring bases, a modified backbone, e.g., a phosphorothioate backbone that promotes stability of the nucleic acid, or both. The nucleic acid advantageously may be incorporated in an expression cassette, vector or plasmid of the invention, comprising features that promote desired expression, replication, and/or selection in target host cell(s). Examples of such features include an origin of replication component, a selection gene component, a promoter component, an enhancer element component, a polyadenylation sequence component, a termination component, and the like, numerous suitable examples of which are known.

The present disclosure further provides the recombinant DNA constructs comprising one or more of the nucleotide sequences described herein. These recombinant constructs are used in connection with a vector, such as a plasmid, phagemid, phage or viral vector, into which a DNA molecule encoding any disclosed antibody is inserted.

Monoclonal antibodies are produced using any method that produces antibody molecules by cell lines in culture e.g., cultivating recombinant eukaryotic (mammalian or insect) or prokaryotic (bacterial) host cells. Examples of suitable methods for preparing monoclonal antibodies include the hybridoma methods of Kohler et al. (1975, Nature 256:495-497) and the human B-cell hybridoma method (Kozbor, 1984, J. Immunol. 133:3001; and Brodeur et al., 1987, Monoclonal Antibody Production Techniques and Applications, (Marcel Dekker, Inc., New York), pp. 51-63).

Antibodies of the present invention may be identified or obtained employing a hybridoma method. In such method, a mouse or other appropriate host animal, such as a hamster, is immunized to elicit lymphocytes that produce or are capable of producing antibodies that will specifically bind to the protein used for immunization. Alternatively, lymphocytes may be immunized in vitro. Lymphocytes then are fused with myeloma cells using a suitable fusing agent, such as polyethylene glycol, to form a hybridoma cell.

Culture medium in which hybridoma cells are growing is assayed for production of monoclonal antibodies directed against the antigen. Preferably, the binding specificity of monoclonal antibodies produced by hybridoma cells is determined by immunoprecipitation or by an in vitro binding assay, such as radioimmunoassay (RIA) or enzyme-linked immunoabsorbent assay (ELISA).

mAbs may then be purified from hybridoma supernatants for further testing for its specific binding of the O3b-antigen and possibly for its differential binding affinity to preferentially bind the O3b-antigen relative to any other O3- or O-antigen, and engineering of antibodies e.g., for different diagnostic or therapeutic purposes.

O3b-specific antibodies, in some instances, emerge through screening against the single O3b-antigen. To increase the likelihood of isolating differentially binding clones one would apply multiple selective pressures by processively screening against the different antigens. Special mAb selection strategies employ the O3b and O3a or O3 components or other K. pneumoniae antigens in an alternating fashion.

Screening methods for identifying antibodies with the desired selective binding properties may be done by display technologies using a library displaying antibody sequences or antigen-binding sequences thereof (e.g., using phage, bacterial, yeast or mammalian cells; or in vitro display systems translating nucleic acid information into respective (poly)peptides). Reactivity can be assessed based on ELISA, Western blotting or surface staining with flow cytometry e.g., using standard assays.

Isolated antigen(s) may e.g., be used for selecting antibodies from an antibody library e.g., a yeast-displayed antibody library.

For example, the invention specifically provides for O3b specific antibodies, which are obtained by a process to identify antibodies with specificities to bind the O3b-antigen e.g., by a specific discovery selection scheme. Accordingly, an antibody library including antibodies showing reactivity with the O3b target, may be selected for reactivity with the target.

The invention moreover provides pharmaceutical compositions which comprise an antibody as described herein and a pharmaceutically acceptable carrier or excipient. These pharmaceutical compositions can be administered in accordance with the present invention as a bolus injection or infusion or by continuous infusion. Pharmaceutical carriers suitable for facilitating such means of administration are well known in the art.

Pharmaceutically acceptable carriers generally include any and all suitable solvents, dispersion media, coatings, antibacterial and antifungal agents, isotonic and absorption delaying agents, and the like that are physiologically compatible with an antibody or related composition or combination provided by the invention. Further examples of pharmaceutically acceptable carriers include sterile water, saline, phosphate buffered saline, dextrose, glycerol, ethanol, and the like, as well as combinations of any thereof.

In one such aspect, an antibody can be combined with one or more carriers appropriate a desired route of administration, antibodies may be e.g., admixed with any of lactose, sucrose, starch, cellulose esters of alkanoic acids, stearic acid, talc, magnesium stearate, magnesium oxide, sodium and calcium salts of phosphoric and sulphuric acids, acacia, gelatin, sodium alginate, polyvinylpyrrolidine, polyvinyl alcohol, and optionally further tableted or encapsulated for conventional administration. Alternatively, an antibody may be dissolved in saline, water, polyethylene glycol, propylene glycol, carboxymethyl cellulose colloidal solutions, ethanol, corn oil, peanut oil, cotton-seed oil, sesame oil, tragacanth gum, and/or various buffers. Other carriers, adjuvants, and modes of administration are well known in the pharmaceutical arts. A carrier may include a controlled release material or time delay material, such as glyceryl monostearate or glyceryl distearate alone or with a wax, or other materials well known in the art.

Additional pharmaceutically acceptable carriers are known in the art and described in, e.g., REMINGTON'S PHARMACEUTICAL SCIENCES. Liquid formulations can be solutions, emulsions or suspensions and can include excipients such as suspending agents, solubilizers, surfactants, preservatives, and chelating agents.

Pharmaceutical compositions are contemplated wherein an antibody of the present invention and one or more therapeutically active agents are formulated. Stable formulations of the antibody of the present invention are prepared for storage by mixing said immunoglobulin having the desired degree of purity with optional pharmaceutically acceptable carriers, excipients or stabilizers, in the form of lyophilized formulations or aqueous solutions. The formulations to be used for in vivo administration are specifically sterile, preferably in the form of a sterile aqueous solution. This is readily accomplished by filtration through sterile filtration membranes or other methods. The antibody and other therapeutically active agents disclosed herein may also be formulated as immunoliposomes, and/or entrapped in microcapsules.

Administration of the pharmaceutical composition comprising an antibody of the present invention, may be done in a variety of ways, including orally, subcutaneously, intravenously, intranasally, intraotically, transdermally, mucosal, topically, e.g., gels, salves, lotions, creams, etc., intraperitoneally, intramuscularly, intrapulmonary e.g., employing inhalable technology or pulmonary delivery systems, vaginally, parenterally, rectally, or intraocularly.

Examplary formulations as used for parenteral administration include those suitable for subcutaneous, intramuscular or intravenous injection as, for example, a sterile solution, emulsion or suspension.

In one embodiment, the antibody of the present invention is the only therapeutically active agent administered to a subject e.g., as a disease modifying or preventing monotherapy.

In another embodiment, the antibody of the present invention is combined with further antibodies in a cocktail e.g., combined in a mixture or kit of parts, to target Klebsiella pneumoniae, such that the cocktail contains more than one therapeutically active agents administered to a subject e.g., as a disease modifying or preventing combination therapy.

Further, the antibody of the present invention may be administered in combination with one or more other therapeutic or prophylactic agents, including but not limited to standard treatment e.g., antibiotics, steroid and non-steroid inhibitors of inflammation, and/or other antibody based therapy e.g., employing anti-bacterial or anti-inflammatory agents.

A combination therapy is particularly employing a standard regimen e.g., as used for treating infection by Klebsiella pneumoniae. This may include antibiotics, e.g., tygecycline, colistin, polymixin B, and beta lactams with or without non-beta lactam inhibitors.

In a combination therapy, the antibody may be administered as a mixture, or concomitantly with one or more other therapeutic regimens e.g., either before, simultaneously or after concomitant therapy.

The biological properties of the antibody or the respective pharmaceutical preparations of the invention may be characterized ex vivo in cell, tissue, and whole organism experiments. As is known in the art, drugs are often tested in vivo in animals, including but not limited to mice, rats, rabbits, dogs, cats, pigs, and monkeys, in order to measure a drug's efficacy for treatment against a disease or disease model, or to measure a drug's pharmacokinetics, pharmacodynamics, toxicity, and other properties. The animals may be referred to as disease models. Therapeutics are often tested in mice, including but not limited to nude mice, SCID mice, xenograft mice, and transgenic mice (including knockins and knockouts). Such experimentation may provide meaningful data for determination of the potential of the antibody to be used as a therapeutic or as a prophylactic with the appropriate half-life, effector function, (cross-) neutralizing activity and/or immune response upon active or passive immunotherapy. Any organism, preferably mammals, may be used for testing. For example because of their genetic similarity to humans, primates, monkeys can be suitable therapeutic models, and thus may be used to test the efficacy, toxicity, pharmacokinetics, pharmacodynamics, half-life, or other property of the subject agent or composition. Tests in humans are ultimately required for approval as drugs, and thus of course these experiments are contemplated. Thus, the antibody and respective pharmaceutical compositions of the present invention may be tested in humans to determine their therapeutic or prophylactic efficacy, toxicity, immunogenicity, pharmacokinetics, and/or other clinical properties.

The antibody designated 2F8-G6, specifically the antibody light chain and/or heavy chain, is characterized by the biological material deposited at the DSMZ-Deutsche Sammlung von Mikroorganismen und Zellkulturen, Mascheroder Weg 1b/Inhoffenstraße 7B, 38124 Braunschweig (DE) under the accession numbers as indicated herein.

DSM 32059 is an E. coli host cell transformed with a plasmid comprising the coding sequence of the 2F8-G6 VH: Escherichia coli 2F8-G6 VH=DSM 32059, deposition date: Jun. 4, 2015; depositor: Arsanis Biosciences GmbH, Vienna, Austria.

DSM 32060 is an E. coli host cell transformed with a plasmid comprising the coding sequence of the 2F8-G6 VL: Escherichia coli 2F8-G6 VL=DSM 32060; deposition date: Jun. 4, 2015; depositor: Arsanis Biosciences GmbH, Vienna, Austria. The subject matter of the following definitions is considered embodiments of the present invention:

1. An isolated antibody that specifically recognizes an epitope of the lipopolysaccharide (LPS) O3b-antigen structure of Klebsiella pneumoniae, which is a O3b-epitope incorporated in O3b-antigen comprising the structure of Formula (I), including one or more O3b-antigen mannose homopolymer repeating units, wherein Formula (I) is:

MeP→3)-α-D-Manp-(1→2)-α-D-Manp-(1→3)-α-D-Manp-(1→[3)-α-D-Manp-(1→2)-α-D-Manp-(1→3)-α-D-Manp-(1→]ₙ

wherein

MeP is methyl phosphate; and

n is 0-50.

2. The antibody of definition 1, which cross-reacts with an O3a-epitope and/or an O3-epitope, wherein

a) the O3a-epitope is incorporated in the LPS O3a-antigen of Klebsiella pneumoniae comprising the structure of Formula (II), including one or more O3a-antigen mannose homopolymer repeating units, wherein Formula (II) is:

MeP→3)-α-D-Manp-(1→2)-α-D-Manp-(1→2)-α-D-Manp-(1→3)-α-D-Manp-(1→[3)-α-D-Manp-(1→2)-α-D-Manp-(1→2)-α-D-Manp-(1→3)-α-D-Manp-(1]ₘ

wherein m is 0-50;

and

b) the O3-epitope is incorporated in the LPS O3-antigen of Klebsiella pneumoniae comprising the structure of Formula (III), including one or more O3-antigen mannose homopolymer repeating units, wherein Formula (III) is:

MeP→3)-α-D-Manp-(1→2)-α-D-Manp-(1→2)-α-D-Manp-(1→2)-α-D-Manp-(1→3)-α-D-Manp-(1→[3)-α-D-Manp-(1→2)-α-D-Manp-(1→2)-α-D-Manp-(1→2)-α-D-Manp-(1→3)-α-D-Manp-(1→]ₘ

wherein m is 0-50.

3. The antibody of definition 2, which is a pan-O3 specific antibody, specifically binding to the O3b-epitope and cross-reacting with the O3a-epitope and the O3-epitope.

4. The antibody of definition 3, which is the 2F8-G6 antibody or which competitively binds to its specific epitope, wherein the 2F8-G6 antibody is characterized by

a) the VH incorporated in the deposited material DSM 32059; and

b) light VL incorporated in the deposited material DSM 32060.

5. The antibody of definition 3, which is the 4D3-A4 antibody or which competitively binds to its specific epitope, wherein the 4D3-A4 antibody is characterized by any of

a) the 6 CDR sequences which are CDR1, 2, 3, 4, 5, and 6 identified by SEQ ID 1, 2, 3, 4, 5, and 6, respectively, wherein numbering is according to Kabat; or CDR1, 2, 3, 4, 5, and 6 identified by SEQ ID 7, 8, 9, 10, 11, and 12, respectively, wherein numbering is according to IMGT; and/or

b) the VH and VL sequences which are the VH sequence identified by SEQ ID 15 and the VL sequence identified by SEQ ID 16; and/or

c) the HC and LC sequences which are the HC sequence identified by SEQ ID 13 and the LC sequence identified by SEQ ID 14.

6. The antibody of definition 1, which preferentially binds to the O3b-epitope relative to an O3a-epitope, or which does not cross-react with an O3a-epitope, wherein the O3a-epitope is incorporated in O3a-antigen repeating units of the LPS O3a-antigen structure of Klebsiella pneumoniae, wherein the O3a-antigen repeating unit is a mannose homopolymer of Formula (II).

7. The antibody of definition 1, which preferentially binds to the O3b-epitope relative to an O3-epitope, or which does not cross-react with an O3-epitope, wherein the O3-epitope is incorporated in O3-antigen repeating units of the LPS O3-antigen structure of Klebsiella pneumoniae, wherein the O3-antigen repeating unit is a mannose homopolymer of Formula (III).

8. The antibody of definition 6 or 7, which does not cross-react with any of the O3a-epitope and the O3-epitope.

9. The antibody of any of definitions 1 to 8, which does not cross-react with an epitope of non-O3 LPS molecules of Klebsiella pneumoniae.

10. The antibody of any of definitions 1 to 9, which has an affinity to bind the O3b-epitope with a Kd of less than 10⁻⁷ M, preferably less than 10⁻⁸ M, even more preferably less than 10⁻⁹ M.

11. The antibody of any of definitions 1 to 10, which is neutralizing endotoxin of Klebsiella pneumoniae strains expressing O3b LPS molecules.

12. The antibody of any of definitions 1 to 11, which is a full-length monoclonal antibody, an antibody fragment thereof comprising at least one antibody domain incorporating the binding site, or a fusion protein comprising at least one antibody domain incorporating the binding site, specifically wherein the antibody is a non-naturally occurring antibody which comprises a randomized or artificial amino acid sequence.

13. The antibody of any of definitions 1 to 12, which is of human, humanized, chimeric, or murine origin.

14. The antibody of any of definitions 1 to 13, which is a monoclonal antibody.

15. The antibody of any of definitions 1 to 14, for use in treating a subject at risk of or suffering from Klebsiella pneumoniae infection or colonization comprising administering to the subject an effective amount of the antibody to limit the infection in the subject or to ameliorate a disease condition resulting from said infection, preferably for treatment or prophylaxis of any of primary and secondary bacteremia, pneumonia, urinary tract infection, liver abscess, peritonitis, or meningitis.

16. A pharmaceutical preparation comprising the antibody of any of definitions 1 to 14, preferably comprising a parenteral or mucosal formulation, optionally containing a pharmaceutically acceptable carrier or excipient.

17. A use of the antibody of any of definitions 1 to 14, for diagnosis of Klebsiella pneumoniae infection or colonization, or an associated disease such as primary and secondary bacteremia, pneumonia, urinary tract infection, liver abscess, peritonitis, or meningitis in a subject.

18. The use according to definition 17, wherein the subject is an immunocompromised or immunosuppressed patient, or a contact thereof.

19. A diagnostic preparation of the antibody of any of definitions 1 to 14, comprising the antibody and a further diagnostic reagent in a composition or a kit of parts, comprising the components
a) the antibody of any of definitions 1 to 14; and
b) the further diagnostic reagent;
c) and optionally a solid phase to immobilize at least one of the antibody and the diagnostic reagent.

20. The diagnostic preparation of definition 19, wherein the further diagnostic reagent is a diagnostic label or a reagent specifically reacting with the antibody and/or the reaction product of the antibody binding to its antigen.

21. A method of diagnosing Klebsiella pneumoniae infection or colonization in a subject caused by a Klebsiella pneumoniae strain, comprising
a) providing the antibody of any of definitions 1 to 14; and
b) detecting if the antibody specifically immunoreacts with the O3b-epitope in a biological sample of the subject to be tested, thereby diagnosing Klebsiella pneumoniae infection or colonization.

22. The method of definition 21, wherein the biological sample is a body fluid or tissue sample, preferably a sample selected from the group consisting of a blood sample, stool sample, skin sample, urine sample, cerebrospinal fluid, and a respiratory tract specimen such as endotracheal aspirates, pleural fluid, lung tap, nasal swab or sputum, or a Klebsiella pneumoniae isolate originating from any of the foregoing.

23. An isolated nucleic acid encoding the antibody of any of definitions 1 to 14.

25. An expression cassette or a plasmid comprising a coding sequence to express a proteinaceous construct comprising a VH and/or VL of the antibody of any of definitions 1 to 14.

25. A host cell comprising the expression cassette or a plasmid of definition 24.

26. A method of producing the antibody of any of definitions 1 to 14, wherein the host cell of claim 25 is cultivated or maintained under conditions to produce said antibody.

27. A method of identifying a candidate antibody comprising:
a) providing a sample containing an antibody or antibody-producing cell; and
b) assessing for binding of an antibody in or produced by the sample with a O3b-epitope as defined in definition 1, wherein a positive reaction between the antibody and the epitope identifies the antibody as candidate antibody.

28. A method of identifying a candidate antibody comprising:
a) providing a sample containing an antibody or antibody-producing cell; and
b) assessing for binding of an antibody in or produced by the sample with the O3b-epitope as defined in definition 1, wherein a specific positive reaction between the antibody and the O3b-epitope relative to the O3a-epitope and/or O3-epitope as defined in definition 2, or relative to any non-O3 LPS molecule of Klebsiella pneumoniae identifies the antibody as candidate antibody.

29. A method of producing the antibody of any of definitions 1 to 14, comprising
a) providing the candidate antibody identified according to definition 27 or 28; and
b) producing a monoclonal antibody, or a humanized or human form of the candidate antibody, or a derivative thereof with the same epitope binding specificity as the candidate antibody.

The present invention is further illustrated by the following examples without being limited thereto.

### EXAMPLES

### Example 1: Identification of Different O-Antigen Repeat Unit Size Among K. pneumoniae Strains of the O3 Serogroup

LPS was purified with a commercial LPS purification kit (Intron) from different O3 strains (PCM-11 O3:K11 from IITD PAN Wroclaw, Polish Collection of Microorganisms; Kp14, Kp62, Kp18 and Kp35 from clinical collections as well as unrelated serotypes. ˜1 μg LPS was separated by SDS-PAGE and stained with ProQ® Emerald 300 lipopolysaccharide staining kit (LifeTechnologies).

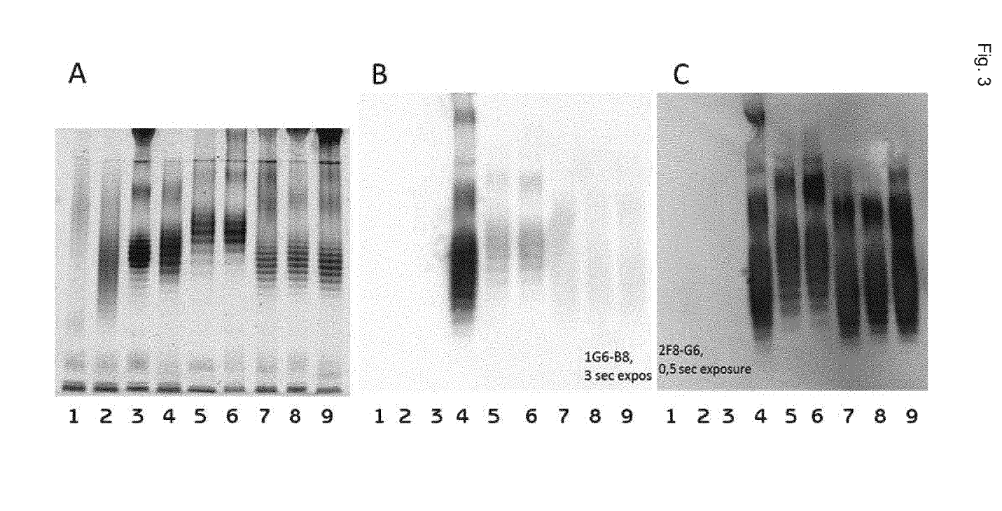

LPS obtained from strains coming from clinical samples and commercial collections were separated and stained. Interestingly, as visualized on FIG. 2, the LPS pattern showed significant variability. Three distinct types could be distinguished that differed not only the modal length of O-antigens (i.e. the average number of O-antigen subunits) but also in the separation of the “ladder-steps”. Given that in this typical ladder-like structure the individual “ladder steps” represent one single O-antigen repeat unit difference, the distance between the ladder steps is characteristic for the size of the repeating units. Apparently, the three distinct LPS molecules within the O3 serogroup exhibit different sizes of their repeating units: lane 2 and 3>lane 1>lanes 4 and 5. Based on this, we proposed that 3 variants of the O3 serogroup exist that differ in the number of mannose molecules within the O-antigen repeating units.

### Example 2: Identification of mAbs Cross-Reacting with all O3 Serotypes

Balb/c mice were immunized intravenously with 10⁷ CFU live K. pneumoniae prototype strain PCM-11 (O3:K11, IITD PAN Wroclaw, Polish Collection of Microorganisms) 3-times with 2-week intervals. Mouse hybridomas were generated by standard procedures and clones secreting specific murine mAbs to purified O-polysaccharide deriving from the immunizing strain were selected.

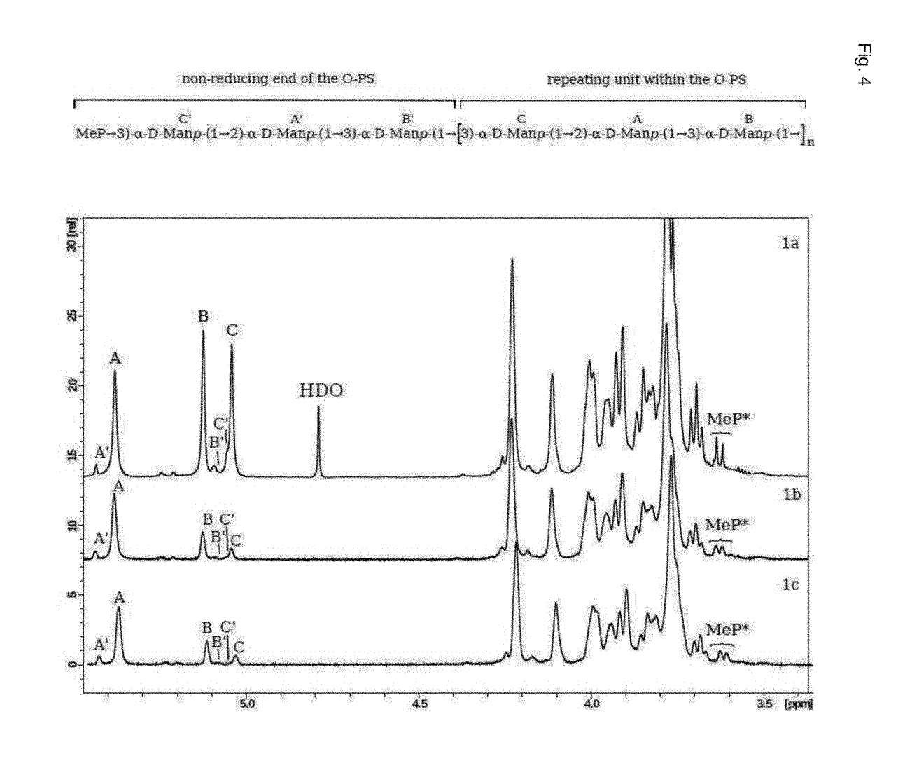

LPS samples separated as described above were transferred to PVDF membrane for immunoblotting. Membranes were reacted with 1 g/ml of murine mAbs and secondary HRP-labelled goat anti-mouse IgG. Blots were developed by ECL reagent. O3-specific mAbs selected from mice immunized with PCM-11 (lane 4 in FIG. 3) showed different reactivity patters. Some mAbs represented by 1G6-B8 (FIG. 3B) reacted mostly with the immunizing strain, however, much less with the other O3 strains showing a different staining pattern (FIGS. 2 and 3A). In contrast other mAbs represented by 2F8-G6 reacted comparably to all O3 variants (FIG. 3C). None of these mAbs stained any of the non-O3 LPS molecules, such as O1, O2, and O5 (lanes 1, 2, and 3, respectively).

These findings confirm that the different types implied by the dislike separation pattern indeed represent antigenically different structures. On the other hand, the different types share common epitopes as well, that was corroborated by the selection of pan-O3 cross-reactive mAbs.

### Example 3: Structural Characterization of the Novel Klebsiella pneumoniae 0 Antigen

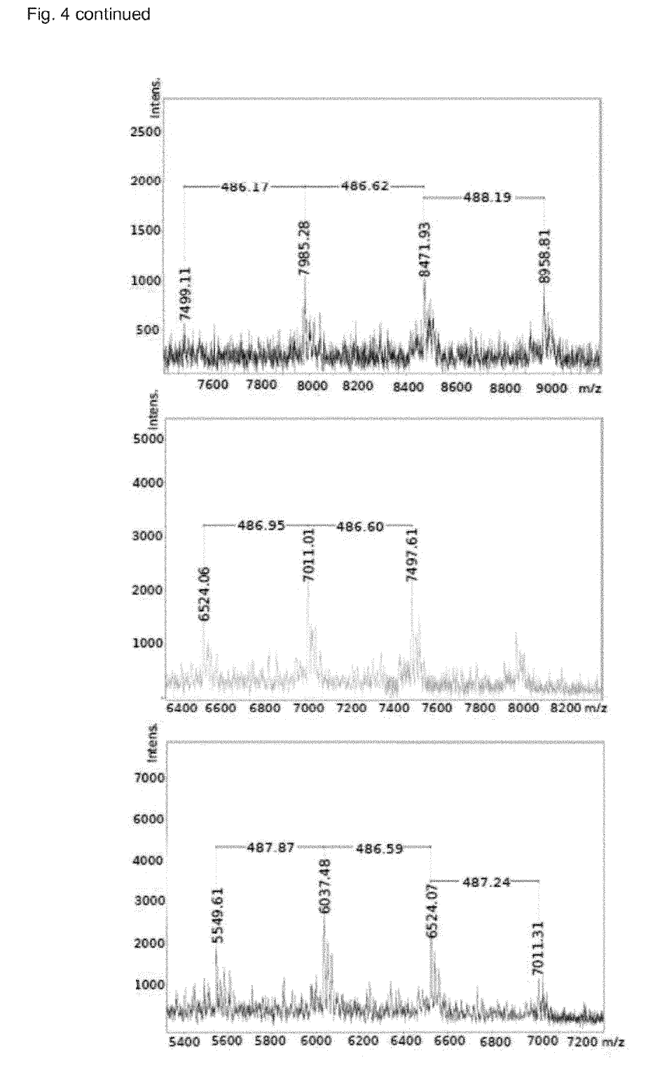

A K. pneumoniae O3b strain Kp81 (clinical isolate) was cultured in LB medium in 10 L fermentor (37° C., 12 h, agitation of 200 rpm, gas flow of 5 L/min), treated with 1% phenol for 2 h at 60° C., centrifuged, washed, resuspended in water, and freeze-dried. The LPS of K. pneumoniae Kp81 was isolated by the hot phenol/water method and purified by dialysis and ultracentrifugation (9). Filtration through glass wool filter was added as an additional step between dialysis and ultracentrifugation. The O-specific polysaccharides (O-PS) and different oligosaccharide components were released by mild acidic hydrolysis (1.5% CH₃COOH, 20 min., 100° C.). Water-soluble poly- and oligosaccharides were ultracentrifuged (6 h, 105000×g, 4° C.) to remove remains of capsular antigen (K antigen, CPS). Obtained poly- and oligosaccharides were fractionated by gel filtration on Bio-Gel P-10 (−400 mesh) (Biorad, USA) or HW-40F (Tosoh Bioscience LLC, USA) yielding fractions 1a, 1b, 1c, 2, 3 and 4. All fractions were analysed by NMR spectroscopy and/or MALDI-TOF mass spectrometry (MS), showing the presence of O-PS in fractions 1a-c (FIG. 4). These fractions contained different number of O-PS repeating units (RU) linked to a primer adaptor sequence and common part of the outer core oligosaccharide with the following general structure: Kdo-GIcNAc-Man-[RU]n.

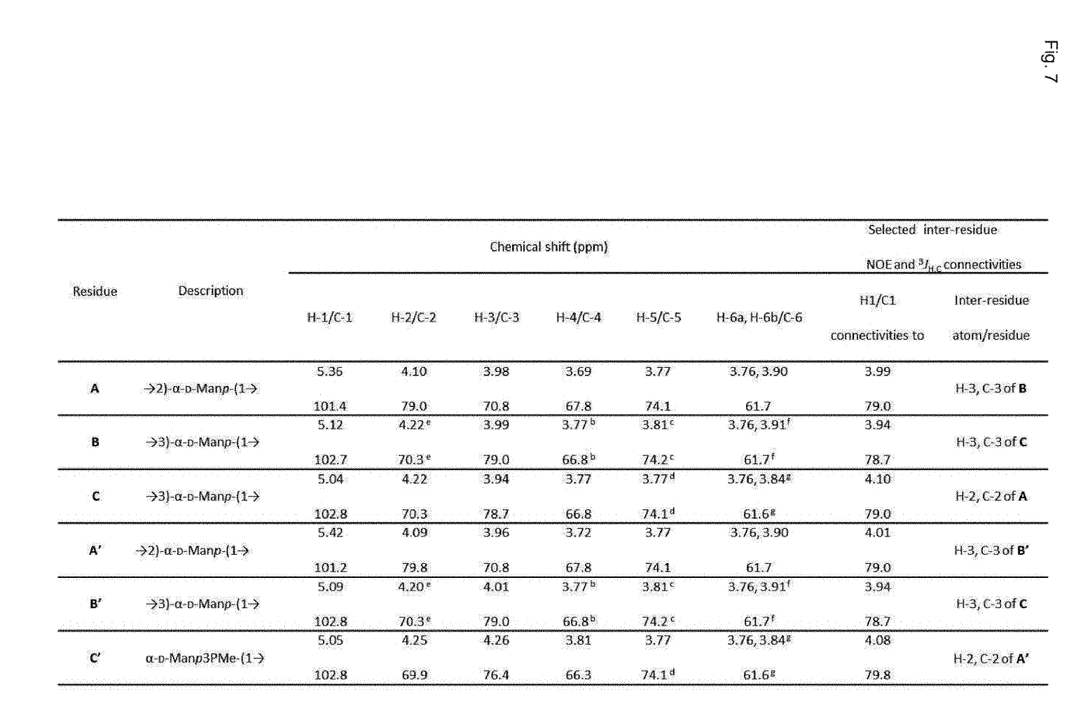

The structure of the repeating unit (RU) of the LPS O-PS from strain Kp81 was determined for the fraction 1a with the use of sugar and methylation analysis, ¹H and ¹³C NMR spectroscopy, and MALDI-TOF MS. Combined results of sugar and methylation analysis confirmed the presence of three prevailing residues: 2- and 3-substituted mannopiranoses (Manp). The complete assignment of the O-PS Kp81 ¹H and ¹³C resonances (FIG. 7) was achieved by interpretation of COSY, TOCSY and NOESY, as well as HSQC-DEPT, HMBC, and HSQC-TOCSY experiments. The ¹H, ¹³C HSQC-DEPT spectrum contained signals for three major anomeric protons and carbons (residues A, B, C) and minor spin systems (residues A′, B′, C′) (FIG. 7, Table 1). Additionally, methyl phosphate (MeP) was identified on the basis of the presence of two sharp proton signals of methyl group (δ_{H} 3.61, 3.63; δ_{C} 53.7 ppm) as a doublet with J_{P,H} of 11.0 Hz indicating its substitution by phosphate group (P). The inter-residue connectivities between adjacent sugar residues were observed by NOESY and HMBC experiments (FIG. 7). The major set of signals was attributed to trisaccharide structure of the Kp81 O-PS biological RU: [→3)-α-D-Manp-(1→2)-α-D-Manp-(1→3)-α-D-Manp-(1→]ₙ ([→3)-C-(1→2)-A-(1→3)-B-(1→]ₙ) (FIG. 4, inset structure). ¹H, ³¹P HMBC spectrum showed correlation of MeP phosphate group to H-3 of residue C′ (α-D-Manp3PMe). Additionally ³¹P, ¹H HMQC-TOCSY showed correlation of MeP phosphate group to H-2, H-3, H-4, H-5 of residue C′, supporting substitution of C′ by MeP. Furthermore, residues A′ (→2)-α-D-Manp) and B′ (→3)-α-D-Manp) were identified and together with C′ were found to be constituents of non-reducing end of the Kp81 O-PS. Complete structure of the Kp81 O-PS including non-reducing end is presented in FIG. 1 and has the following schematic sequence: C′-(1→2)-A′-(1→3)-B′-(1 [→3)-C-(1→2)-A-(1→3)-B-(1→]ₙ.

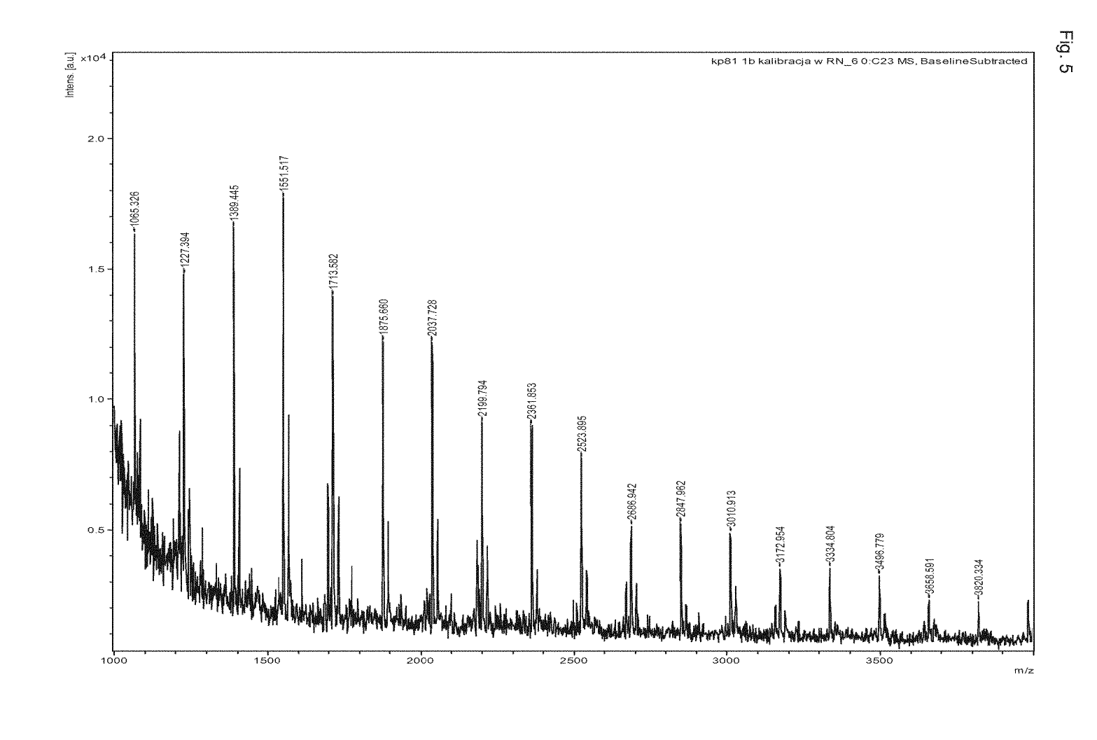

Molecular weight of O-PS Kp81 repeating unit (trisaccharide of Manp) was confirmed with the use of MALDI-TOF MS. Mass spectra of fractions 1a, 1b, 1c showed clusters of ions attributed to different forms of Kdo-GIcNAc-Man-[RU]ₙ. ([M-H]⁻, dehydrated forms, including heptose and MeP substitutions). Average monoisotopic mass difference among these ions was 486.49 Da and matched the mass calculated on the basis of NMR results—486.42 Da. Additionally mass spectrum of the fraction 1b (FIG. 5) contained [M-H₂O-H]⁻ ions (e.g., m/z 1227.39, 1389.45, 1551.52, 1713.58, 1875.66, 2037.73, 2199.79, 2361.85, 2523.90) attributing to O-PS fragments built of MeP-[Man]ₙ. The mass difference between ions of 162 Da was attributed to Man residue. For example the ion at m/z 1713.58 was attributed to MeP-[Man]₁₀ polymer. These fragments resulted from MALDI in-source fragmentation of native O-PS and indicates the presence of one MeP group per O-PS.

### Example 4: Surface Binding by LPS-O3 Cross-Reactive mAb

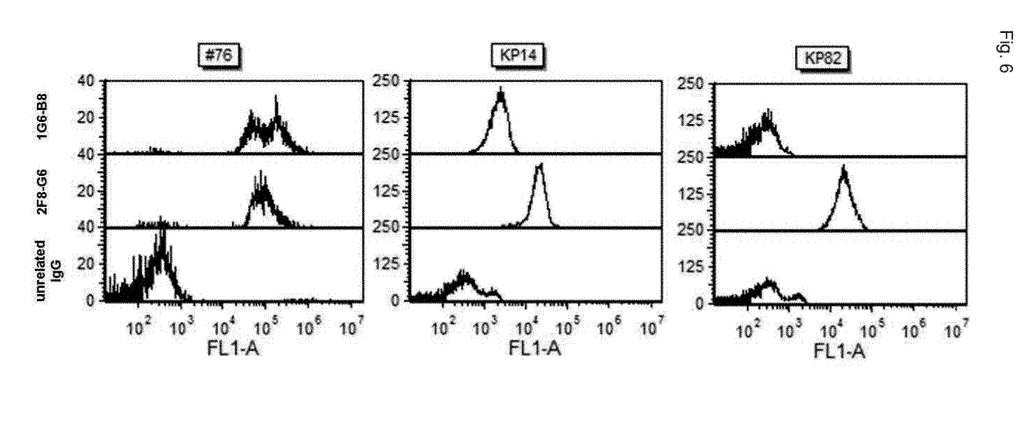

Surface staining by the mAbs of different mannan specificity (FIG. 6) on live Klebsiella strains was investigated by flow cytometry. Washed bacteria (10⁶ CFU) were reacted with O3 specific mAbs (40 μg/ml) followed by staining with 2 μg/ml of Alexa Fluor® 488-conjugated goat anti-human IgG secondary antibody (Life Technologies) and 5 μM SYTO-62 nucleic acid stain (Life Technologies). Samples were quantified in a BD AccuriTM C6 flow cytometer (BD Biosciences) and data were analyzed using the FCS Express software version 4 (De Novo Software).

The cross-reactive mAb 2F8-G6 bound strongly to the surface of all O3 Klebsiella strains investigated irrespective of the mannan structure of the O-antigens expressed. In contrast, a comparable mAb 1G6-B8 stained strongly the O3a immunizing strain (PCM-11), only. It bound weakly onto the surface of strain Kp14 expressing O3, and not at all to strain Kp82 expressing O3b antigens.

Flow cytometry analysis, therefore, confirmed that surface staining of corresponding live Klebsiella strains by mannan specific mAbs is determined exclusively by antigen specificity, and not restricted by the various smooth LPS molecules. Consequently, antibodies with broad specificity are promising candidates for further development as potential antibacterial agents.

### Example 5: Cross-Neutralization of Endotoxin Activities of O3 Type LPS Molecules

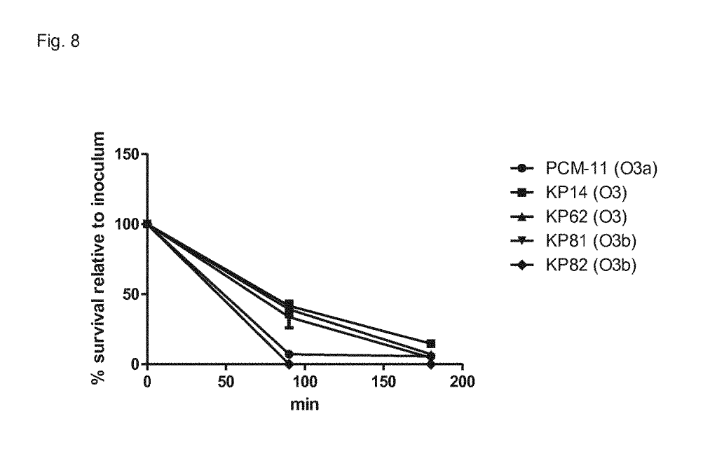

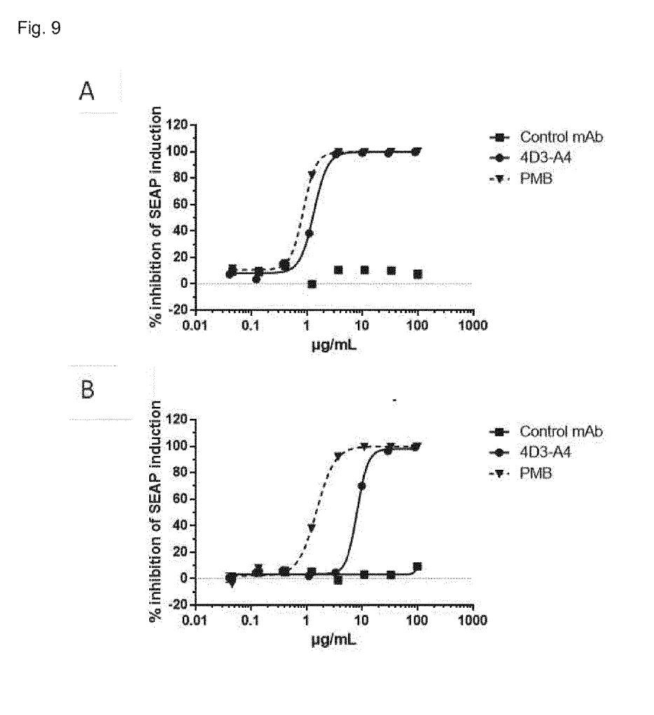

Since K. pneumoniae O3 strains are sensitive to the bactericidal effect of normal human serum (FIG. 8), a clinically relevant mode of action for prospective therapeutic mAbs is the anti-inflammatory potential of such mAbs. Endotoxin (i.e. the Lipid A part of the LPS molecules) released from the lysed bacteria trigger a strong inflammatory response through signaling via its innate receptor complex TLR-4/CD14/MD2. The impact of mAb 4D3-A4 on the signaling elicited by different K. pneumoniae O3 type LPS molecules was investigated in a cell based in vitro system (HEK Blue, InvivoGen). Signaling through the human TLR-4 complex expressed by the cells is translated to a colorimetric signal. FIG. 9 shows that mAb 4D3-A4 could inhibit the signaling, i.e. neutralize the endotoxic effect of extracted purified LPS molecules. Importantly, dose dependent neutralization was observed for different O3 subtypes, i.e. the O3 cross-reactive mAb was comparably potent against LPS extracted from either O3a (strain PCM-11 from IITD PAN Wroclaw, Polish Collection of Microorganisms FIG. 9A) or O3b (strain Kp81 clinical isolate from clinical collection; FIG. 9B).

### REFERENCES

1. Trautmann M, Ruhnke M, Rukavina T, Held T K, Cross A S, Marre R, Whitfield C. 1997. O-antigen seroepidemiology of Klebsiella clinical isolates and implications for immunoprophylaxis of Klebsiella infections. Clin. Diagn. Lab Immunol. 4:550-555.
2. Hansen D S, Mestre F, Alberti S, Hernandez-Alles S, Alvarez D, Domenech-Sanchez A, Gil J, Merino S, Tomas J M, Benedi V J. 1999. Klebsiella pneumoniae lipopolysaccharide O typing: revision of prototype strains and O-group distribution among clinical isolates from different sources and countries. J. Clin. Microbiol. 37:56-62.
3. Vinogradov E, Frirdich E, MacLean L L, Perry M B, Petersen B O, Duus J O, Whitfield C. 2002. Structures of lipopolysaccharides from Klebsiella pneumoniae. Eluicidation of the structure of the linkage region between core and polysaccharide O chain and identification of the residues at the non-reducing termini of the O chains. J. Biol. Chem. 277:25070-25081.
4. Kubler-Kielb J, Whitfield C, Katzenellenbogen E, Vinogradov E. 2012. Identification of the methyl phosphate substituent at the non-reducing terminal mannose residue of the O-specific polysaccharides of Klebsiella pneumoniae O3, Hafnia alvei PCM 1223 and Escherichia coli O9/O9a LPS. Carbohydr. Res. 347:186-188.
5. Greenfield L K, Richards M R, Li J, Wakarchuk W W, Lowary T L, Whitfield C. 2012. Biosynthesis of the polymannose lipopolysaccharide O-antigens from Escherichia coli serotypes O8 and O9a requires a unique combination of single- and multiple-active site mannosyltransferases. J. Biol. Chem. 287:35078-35091.
6. Greenfield L K, Richards M R, Vinogradov E, Wakarchuk W W, Lowary T L, Whitfield C. 2012. Domain organization of the polymerizing mannosyltransferases involved in synthesis of the Escherichia coli O8 and O9a lipopolysaccharide O-antigens. J. Biol. Chem. 287:38135-38149.
7. Kido N, Kobayashi H. 2000. A single amino acid substitution in a mannosyltransferase, WbdA, converts the Escherichia coli O9 polysaccharide into O9a: generation of a new O-serotype group. J. Bacteriol. 182:2567-2573.
8. Parolis L A, Parolis H, Dutton G G. 1986. Structural studies of the O-antigen polysaccharide of Escherichia coli O9a. Carbohydr. Res. 155:272-276.
9. Lukasiewicz J, Dzieciatkowska M, Niedziela T, Jachymek W, Augustyniuk A, Kenne L, Lugowski C. 2006. Complete lipopolysaccharide of Plesiomonas shigelloides O74:H5 (strain CNCTC 144/92). 2. Lipid A, its structural variability, the linkage to the core oligosaccharide, and the biological activity of the lipopolysaccharide. Biochemistry 45:10434-10447.
10. Curvall M, Lindberg B, Lonngren J, Nimmich W. 1973. Structural studies on the Klebsiella O group 3 lipopolysaccharide. Acta Chem. Scand. 27:2645-2649.

1-28. (canceled)

29. An isolated antibody that specifically recognizes an epitope of the lipopolysaccharide (LPS) O3b-antigen structure of Klebsiella pneumoniae, which is a O3b-epitope incorporated in O3b-antigen comprising the structure of Formula (I), including one or more O3b-antigen mannose homopolymer repeating units, wherein Formula (I) is:
MeP→3)-α-D-Manp-(1→2)-α-D-Manp-(1→3)-α-D-Manp-(1→[3)-α-D-Manp-(1→2)-α-D-Manp-(1→3)-α-D-Manp-(1→]ₙ
wherein
MeP is methyl phosphate; and
n is 0-50.

30. The antibody of claim 29, which cross-reacts with an O3a-epitope and/or an O3-epitope, wherein
a) the O3a-epitope is incorporated in the LPS O3a-antigen of Klebsiella pneumoniae comprising the structure of Formula (II), including one or more O3a-antigen mannose homopolymer repeating units, wherein Formula (II) is:
MeP→3)-α-D-Manp-(1→2)-α-D-Manp-(1→2)-α-D-Manp-(1→3)-α-D-Manp-(1→[3)-α-D-Manp-(1→2)-α-D-Manp-(1→2)-α-D-Manp-(1→3)-α-D-Manp-(1→4]ₘ
wherein m is 0-50;
and
b) the O3-epitope is incorporated in the LPS O3-antigen of Klebsiella pneumoniae comprising the structure of Formula (III), including one or more O3-antigen mannose homopolymer repeating units, wherein Formula (III) is:
MeP→3)-α-D-Manp-(1→2)-α-D-Manp-(1→2)-α-D-Manp-(1→2)-α-D-Manp-(1→3)-α-D-Manp-(1→[3)-α-D-Manp-(1→2)-α-D-Man-(1→2)-α-D-Manp-(1→2)-α-D-Manp-(1→3)-α-D-Manp-(1→]ₘ
wherein m is 0-50.

31. The antibody of claim 30, which is a pan-O3 specific antibody, specifically binding to the O3b-epitope and cross-reacting with the O3a-epitope and the O3-epitope.

32. The antibody of claim 31, which is the 2F8-G6 antibody or which competitively binds to its specific epitope, wherein the 2F8-G6 antibody is characterized by
a) the VH incorporated in the deposited material DSM 32059; and
b) light VL incorporated in the deposited material DSM 32060,
which deposited materials were deposited at the DSMZ on Jun. 4, 2015;
preferably wherein competition of binding is determined by competition ELISA analysis or by ForteBio analysis.

33. The antibody of claim 31, which is the 4D3-A4 antibody or which competitively binds to its specific epitope, wherein the 4D3-A4 antibody is characterized by any of
a) the 6 CDR sequences which are CDR1, 2, 3, 4, 5, and 6 identified by SEQ ID 1, 2, 3, 4, 5, and 6, respectively, wherein numbering is according to Kabat; or CDR1, 2, 3, 4, 5, and 6 identified by SEQ ID 7, 8, 9, 10, 11, and 12, respectively, wherein numbering is according to IMGT; and/or
b) the VH and VL sequences which are the VH sequence identified by SEQ ID 15 and the VL sequence identified by SEQ ID 16; and/or
c) the HC and LC sequences which are the HC sequence identified by SEQ ID 13 and the LC sequence identified by SEQ ID 14.

34. The antibody of claim 29, which preferentially binds to the O3b-epitope relative to an O3a-epitope, or which does not cross-react with the O3a-epitope, wherein the O3a-epitope is incorporated in O3a-antigen repeating units of the LPS O3a-antigen structure of Klebsiella pneumoniae, wherein the O3a-antigen repeating unit is a mannose homopolymer of Formula (II), wherein Formula (II) is:
MeP→3)-α-D-Manp-(1→2)-α-D-Manp-(1→2)-α-D-Manp-(1→3)-α-D-Manp-(1→[3)-α-D-Manp-(1→2)-α-D-Manp-(1→2)-α-D-Manp-(1→3)-α-D-Manp-(1→]ₘ
wherein m is 0-50.

35. The antibody of claim 29, which preferentially binds to the O3b-epitope relative to an O3-epitope, or which does not cross-react with the O3-epitope, wherein the O3-epitope is incorporated in O3-antigen repeating units of the LPS O3-antigen structure of Klebsiella pneumoniae, wherein the O3-antigen repeating unit is a mannose homopolymer of Formula (III), wherein Formula (III) is:
MeP→3)-α-D-Manp-(1→2)-α-D-Manp-(1→2)-α-D-Manp-(1→2)-α-D-Manp-(1→3)-α-D-Manp-(1→[3)-α-D-Manp-(1→2)-α-D-Manp-(1→2)-α-D-Manp-(1→2)-α-D-Manp-(1→3)-α-D-Manp-(1→]ₘ
wherein m is 0-50.

36. The antibody of claim 34, which does not cross-react with any of the O3a-epitope and an O3-epitope wherein the O3-epitope is incorporated in O3-antigen repeating units of the LPS O3-antigen structure of Klebsiella pneumoniae, wherein the O3-antigen repeating unit is a mannose homopolymer of Formula (III), wherein Formula (III) is:
MeP→3)-α-D-Manp-(1→2)-α-D-Manp-(1→2)-α-D-Manp-(1→2)-α-D-Manp-(1→3)-α-D-Manp-(1→[3)-α-D-Manp-(1→2)-α-D-Manp-(1→2)-α-D-Manp-(1→2)-α-D-Manp-(1→3)-α-D-Manp-(1→]ₘ
wherein m is 0-50.

37. The antibody of claim 29, which does not cross-react with an epitope of non-O3 LPS molecules of Klebsiella pneumoniae.

38. The antibody of claim 29, which has an affinity to bind the O3b-epitope with a Kd of less than 10⁻⁷M, less than 10⁻⁸M, or less than 10⁻⁹M.

39. The antibody of claim 29, which is neutralizing endotoxin of Klebsiella pneumoniae strains expressing O3b LPS molecules.

40. The antibody of claim 29, which is a full-length monoclonal antibody, an antibody fragment thereof comprising at least one antibody domain incorporating the binding site of the antibody, or a fusion protein comprising at least one antibody domain incorporating the binding site of the antibody, wherein the antibody is a non-naturally occurring antibody which comprises a randomized or artificial amino acid sequence.

41. The antibody of claim 29, which is a monoclonal antibody of human, humanized, chimeric, or murine origin.

42. A method of treating a subject at risk of or suffering from Klebsiella pneumoniae infection or colonization comprising administering to the subject an effective amount of the antibody of claim 29 to limit the infection in the subject or to ameliorate a disease condition resulting from said infection.

43. A pharmaceutical preparation comprising:
the antibody of claim 29,
a parenteral or mucosal formulation, and
optionally a pharmaceutically acceptable carrier or excipient.

44. The method of claim 48, wherein the method is a method of diagnosing an associated disease in the subject selected from the group consisting of: primary and secondary bacteremia, pneumonia, urinary tract infection, liver abscess, peritonitis, and meningitis.

45. The method of claim 48, wherein the subject is an immunocompromised or immunosuppressed patient, or a contact thereof.

46. A diagnostic preparation in a composition or a kit of parts, comprising:
a) the antibody of claim 29;
b) a further diagnostic reagent; and
c) optionally a solid phase to immobilize at least one of the antibody and the diagnostic reagent.

47. The diagnostic preparation of claim 46, wherein the further diagnostic reagent is a diagnostic label or a reagent specifically reacting with the antibody and/or the reaction product of the antibody binding to its antigen.

48. A method of diagnosing Klebsiella pneumoniae infection or colonization in a subject caused by a Klebsiella pneumoniae strain or an associated disease, comprising:
a) providing the antibody of claim 29; and
b) detecting if the antibody specifically immunoreacts with the O3b-epitope in a biological sample of the subject to be tested, thereby diagnosing Klebsiella pneumoniae infection or colonization.

49. The method of claim 48, wherein the biological sample is a body fluid or tissue sample selected from the group consisting of: a blood sample, stool sample, skin sample, urine sample, cerebrospinal fluid, a respiratory tract specimen, endotracheal aspirates, pleural fluid, lung tap, nasal swab, sputum, and a Klebsiella pneumoniae isolate originating from any of the foregoing.

50. An isolated nucleic acid encoding the antibody of claim 29.

51. An expression cassette or a plasmid comprising a coding sequence expressing a proteinaceous construct comprising a VH and/or VL of the antibody of claim 29.

52. A host cell comprising the expression cassette or a plasmid of claim 51.

53. A method of producing the antibody of claim 29 comprising:
cultivating or maintaining a host cell comprising an expression cassette or a plasmid comprising a coding sequence expressing the antibody;
wherein the host cell is cultivated or maintained under conditions to produce said antibody.

54. A method of identifying a candidate antibody according to claim 29 comprising:
a) providing a sample containing an antibody or antibody-producing cell; and
b) assessing for binding of the antibody in or produced by the sample with the O3b-epitope,
wherein a positive reaction between the antibody and the O3b-epitope identifies the antibody as the candidate antibody.

55. A method of identifying a candidate antibody according to claim 29 comprising:
a) providing a sample containing an antibody or antibody-producing cell;
b) assessing for binding of the antibody in or produced by the sample with the O3b-epitope; and
c) assessing for binding of the antibody with one or more of an O3a-epitope, an O3-epitope and any non-O3 LPS molecule of Klebsiella pneumoniae;
wherein the O3a-epitope is incorporated in the LPS O3a-antigen of Klebsiella pneumoniae comprising the structure of Formula (II), including one or more O3a-antigen mannose homopolymer repeating units, wherein Formula (II) is:
MeP→3)-α-D-Manp-(1→2)-α-D-Manp-(1→2)-α-D-Manp-(1→3)-α-D-Manp-(1→[3)-α-D-Manp-(1→2)-α-D-Manp-(1→2)-α-D-Manp-(1→3)-α-D-Manp-(1→]ₘ
wherein m is 0-50;
wherein the O3-epitope is incorporated in the LPS O3-antigen of Klebsiella pneumoniae comprising the structure of Formula (III), including one or more O3-antigen mannose homopolymer repeating units, wherein Formula (III) is:
MeP→3)-α-D-Manp-(1→2)-α-D-Manp-(1→2)-α-D-Manp-(1→2)-α-D-Manp-(1→3)-α-D-Manp-(1→[3)-α-D-Manp-(1→2)-α-D-Manp-(1→2)-α-D-Manp-(1→2)-α-D-Manp-(1→3)-α-D-Manp-(1→]ₘ
wherein m is 0-50; and
wherein a specific positive reaction between the antibody and the O3b-epitope relative to the O3a-epitope, the O3-epitope, or the non-O3 LPS molecule of Klebsiella pneumoniae identifies the antibody as the candidate antibody.

56. A method of producing an antibody, comprising
a) providing the candidate antibody identified according to claim 54; and
b) producing a monoclonal antibody, or a humanized or human form of the candidate antibody, or a derivative thereof with the same epitope binding specificity as the candidate antibody.
